SEQUENCE LISTING (7 sequences)

SEQ ID NO: 1 - protein, 273 aa, Homo sapiens
LTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQL

SEQ ID NO: 2 - DNA, 26 bp, Artificial Sequence
  Sense primer SLC34A2
caccatggctccctggcctgaattgg

SEQ ID NO: 3 - DNA, 34 bp, Artificial Sequence
  Antisense primer for C-terminus of ROS1
ttaatcagacccatctccatatccactgtgagtg

SEQ ID NO: 4 - DNA, 38 bp, Artificial Sequence
  FIG-ROS M13-Kin3F primer
gtaaaacgacggccagtgcgagactagctgccaagtac

SEQ ID NO: 5 - DNA, 38 bp, Artificial Sequence
  ROS M13-Kin1 REV primer
caggaaacagctatgaccgccatcttcaccttcaaagc

SEQ ID NO: 6 - DNA, 18 bp, Artificial Sequence
  M13F primer
gtaaaacgacggccagtg

SEQ ID NO: 7 - DNA, 18 bp, Artificial Sequence
  M13Rev Primer
caggaaacagctatgacc

(19) United States
(12) Patent Application Publication
(10) Pub. No.: US 2014/0243332 A1
(43) Pub. Date: Aug. 28, 2014

(54) METHODS OF TREATING CANCERS CHARACTERIZED BY ABERRENT ROS1 ACTIVITY

(71) Applicants: Monika A. Davare, Portland, OR (US); Brian J. Druker, Portland, OR (US)

(72) Inventors: Monika A. Davare, Portland, OR (US); Brian J. Druker, Portland, OR (US)

(73) Assignee: Oregon Health & Science University, Portland, OR (US)

(21) Appl. No.: 14/190,246

(22) Filed: Feb. 26, 2014

Related U.S. Application Data
(60) Provisional application No. 61/769,936, filed on Feb. 27, 2013.
(60) Provisional application No. 61/901,148, filed on Nov. 7, 2013.

(51) Int. Cl. C12Q 1/68; A61K 31/5377

(52) U.S. Cl. CPC C12Q 1/6874; A61K 31/5377
USPC 514/235.2; 435/6.12; 506/16

(57) ABSTRACT

Disclosed herein are methods of treating cancers characterized by aberrant ROS1 activity by administering an effective amount of foretinib (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide), including methods of identifying mutations in the kinase domain of ROS1 that indicate resistance to crizotinib, but sensitivity to foretinib.

### PRIORITY CLAIM

This application is related to U.S. Provisional Application 61/769,936, filed on 27 Feb. 2013, and U.S. Provisional Application 61/901,148, filed on 7 Nov. 2013, both of which are hereby incorporated by reference in their entireties.

### BACKGROUND

Chromosomal rearrangements of the receptor tyrosine kinase (RTK) ROS1 that create oncogenic fusion kinases are reported in glioma, lung adenocarcinoma and cholangiocarcinoma patients (Bergethon K et al, Off J Am Soc Clin Oncol 30, 863-870 (2012); Charest A et al, Genes, Chrom, Cancer 37, 58-71 (2003); and Gu T L et al, PLoS One 6, e15640 (2011); all of which are incorporated by reference herein). With growing recognition of ROS1 as a therapeutic target, it is necessary to identify ROS1 inhibitors with potential for clinical development Chin L P et al, Off Publ International Assoc for the Study of Lung Cancer 7, 1625-1630 (2012); EI-Deeb I M et al, Med Res Rev (2010); Ou S H et al, Exp Rev Anticancer Therapy 12, 447-456 (2012); all of which are incorporated by reference herein.)

### SUMMARY

A kinase inhibitor screen revealed that foretinib (GSK1363089) is a highly potent ROS1 inhibitor, with selective efficacy for ROS1 compared to the related anaplastic lymphoma kinase (ALK). In vivo, foretinib-treated FIG-ROS-driven tumors exhibited a significant reduction in tumor volume in contrast to treated Pten-null tumors or upon crizotinib treatment of either cohort. Hence foretinib is a more effective ROS1 inhibitor than the FDA-approved inhibitor crizotinib that is now in clinical trial for lung adenocarcinoma patients with ROS1-rearrangements. Patients treated with tyrosine kinase inhibitors regularly develop clinical resistance due in part to emergence of kinase domain (KD) mutations (Shah N P et al, Cancer Cell 2, 117-125 (2002); incorporated by reference herein.) Using an accelerated mutagenesis screen, ROS1 kinase domain mutations were identified that confer resistance to crizotinib. Crizotinib-resistant ROS1 kinase domain mutants retain foretinib sensitivity at concentrations below a level already demonstrated in clinical trials to be safe for human subjects.

Disclosed herein are methods useful in identifying a sample from a subject as comprising a cancer cell that is sensitive to foretinib. In some aspects of the invention, the cell is also identified as resistant to crizotinib. The methods involve isolating a nucleic acid from the sample that includes a nucleic acid that encodes the ROS1 kinase domain (SEQ ID NO: 1) and identifying one or more mutations in the ROS1 kinase domain that results in one or more of the following amino acid substitutions: G2032R, V2098I, G1971E, L1982F, L1982R, C2060G, L1947R or E1935G. The presence of one or more of the mutations identifies the cell as sensitive to foretinib and resistant to crizotinib. The method may further comprise treating the subject with foretinib.

Disclosed herein are kits used to facilitate the performance of the method. The kits include a set of oligonucleotides used to amplify SEQ ID NO: 1 and a set of oligonucleotides used to identify a mutation in SEQ ID NO: 1 that results in one or more of the following amino acid substitutions: G2032R, V2098I, G1971E, L1982F, L1982R, C2060G, L1947R or E1935G.

Disclosed herein are pharmaceutical compositions for use in treating cancers characterized by aberrant activity of the ROS1 kinase domain. The pharmaceutical compositions include foretinib and a pharmaceutically acceptable carrier. The ROS1 kinase domain includes a mutation that results in one or more of the following amino acid substitutions: G2032R, V2098I, G1971E, L1982F, L1982R, C2060G, L1947R or E1935G.

### BRIEF DESCRIPTION OF THE DRAWINGS

Some of the drawings in this disclosure are images that may not reproduce properly in a patent application publication. Additionally, some of the graphs, plots, and photographic images may be better understood using color, which is not available in a patent application publication. Applicants consider all originally disclosed images and graphs (whether in color or not) part of the original disclosure and reserve the right to present high quality and/or color images of the herein described figures in later proceedings.

FIG. 1A is a line graph depicting the viability of Ba/F3 FIG-ROS normalized to a vehicle treated control (control=1.0) cells after 72 hours of exposure to the indicated concentrations of crizotinib, foretinib, Gö6976, TAE684 and GSK1838705A. The symbols represent the mean and the error bars represent the standard error of the mean (SEM) from three independent experiments with triplicate wells.

FIG. 1B is a plot depicting the IC₅₀ values for the Ba/F3 FIG-ROS, Ba/F3 SLC-ROS, Ba/F3 ALK F1174L, Ba/F3 BCR-Abl, Ba/F3 JAK3 A572V and Ba/F3 parental cells treated with the indicated inhibitors. IC₅₀ was determined from non-linear regression curve fit analysis of the dose response curves.

FIG. 1C is an image of an immunoblot of lysates of Ba/F3 FIG-ROS and Ba/F3 SLC-ROS and the indicated downstream effectors after treatment with the indicated compounds. Images shown are representative of three independent experiments.

FIG. 1D is a line graph depicting the viability of HCC78 cells after 72 hour exposure to the indicated concentrations of crizotinib and foretinib. The values are normalized to that of a vehicle treated control (control=1.0). Symbols represent the mean and the error bars represent the standard error of the mean (SEM) from three independent experiments with triplicate wells.

FIG. 1E is an image of an immunoblot of lysates of HCC78 cells probing for phosphorylated and total SLC-ROS1 and ERK1/2, and phosphorylated protein S6 after treatment with the indicated concentration of the indicated inhibitor. The images are representative of two independent experiments.

FIG. 1F top panel is an image of colonies formed by HCC78 cells plated after treatment with the indicated concentration of crizotinib or foretinib after 10 days. FIG. 1F bottom panel is a bar graph depicting the number of colonies observed on the plates from FIG. 1F top panel. Where indicated, **=p<0.01 and ***=p<0.001 by Student's t-test.

FIG. 2A is a plot of the IC₅₀ values for shPten (lines 1 and 2) and FIG-ROS (lines 3 and 4) driven murine cholangiocarcinoma cell lines treated with the indicated inhibitors as determined from non-linear regression curve fit analysis of the dose response curves.

FIG. 2B is a bar graph depicting the anchorage-independent colony formation of FIG-ROS (Line 4) and shPten (Line 2) cholangiocarcinoma cell lines treated with crizotinib and foretinib at the indicated concentrations. The numbers of colonies formed in the presence of crizotinib and foretinib are normalized to a vehicle treated control (control=1.0). The values indicate the mean and the error bars indicate the standard error of the mean of 3 independent wells.

FIG. 2C is an image of an immunoblot of lysates of lines 3 and 4 probed for phosphorylated and total ROS1, phosphorylated Shpt, phosphorylated Erk1/2, total Erk2, and tubulin as a loading control after treatment with the indicated doses of crizotinib and foretinib. Images shown are representative of three independent experiments.

FIG. 3A is a set of 12 images of explanted FIG-ROS and shPten-driven subcutaneous tumors after 9 days of treatment with foretinib (25 mg/kg) or crizotinib (25 mg/kg) by oral gavage.

FIG. 3B is a set of waterfall plots indicating the change in FIG-ROS and shPten tumor volume from treatment initiation to 24 hours after the administration of the last dose. Relative tumor growth was calculated as described in Example 1.

FIG. 3C is a set of images of H&E stained tumor sections from Line 1 (shPten) and Line 3 (FIG-ROS) after 9 days of treatment with vehicle, crizotinib, or foretinib. Scale bar is 500 μm.

FIG. 3D is an image of an immunoblot of tumor lysates probed for phosphorylated and total ROS1, phosphorylated and total Shpt, phosphorylated and total Stat3, phosphorylated and total Src as well as tubulin as a loading control. Tumors were harvested from vehicle or foretinib (25 mg/kg) treated mice the indicated times after oral gavage.

FIG. 4A is a bar graph depicting the percent frequency of the indicated ROS1 kinase domain mutations found in the ENU-treated Ba/F3 FIG-ROS clones isolated after growth and clonal expansion in the presence of 1000 nM crizotinib

FIG. 4B is a structure of a homology model of the ROS1 kinase domain bound to crizotinib (magenta) and foretinib (orange). Foretinib and crizotinib are shown in colored stick representation, while the protein is shown in grey ribbon. Differences in the activation loop conformations are highlighted by coloring them the same as their respective ligands. Residues, which were found to confer resistance to crizotinib when mutated, are highlighted in red and shown in stick representation.

FIG. 4C is a plot of the IC₅₀ values for Ba/F3 FIG-ROS unmutated kinase domain and FIG-ROS kinase domain mutant cells treated with foretinib and crizotinib. IC₅₀ was determined from non-linear regression curve fit analysis of the dose response curves. The dotted lines indicate steady state Cₘₐₓ for crizotinib and foretinib as reported in the literature.

FIG. 4D is a plot of the growth of Ba/F3 CD74-ROS wild-type and G2032R mutant cells after a 72 hour exposure to graded concentrations of crizotinib or foretinib. Data are shown as normalized to vehicle-treated cells. The values are means±SEM from three independent experiments performed in triplicate.

FIG. 4E is a plot of the IC₅₀ values for Ba/F3 CD74-ROS wild-type and G2032R mutant cells treated with foretinib and crizotinib as determined from non-linear regression curve fit analysis of the dose response curves. Dotted lines indicate the previously reported steady state Cₘₐₓ for crizotinib and foretinib as indicated.

FIG. 4F is an image of an Immunoblot analysis of CD74-ROS (wild-type versus G2032R) phosphorylation and downstream effector modulation after treatment of cells with varying doses of crizotinib and foretinib as shown. Cropped images representative of three independent experiments are shown.

FIG. 5A is a set of three bar graphs depicting the induction of apoptosis in Ba/F3 FIG-ROS, Ba/F3 SLC-ROS and Ba/F3 ALK F1174L cells after 48 hours of treatment with the indicated doses of the indicated compounds. The number of cells that were Annexin V and 7-AAD positive after inhibitor treatment was normalized to number of cells staining positive with these markers with vehicle treatment (basal). A minimum of 2000 cells were counted per condition. Rate of apoptosis is shown as fold over basal. Basal apoptosis in Ba/F3 FIG-ROS, Ba/F3 SLC-ROS and Ba/F3 ALK F1174 cells was 3.6%, 2.8% and 1.2% respectively.

FIG. 5B is a bar graph indicating the apoptosis induction in HCC78 cells after treatment with foretinib and crizotinib at the indicated concentrations for 72 hours. Basal (vehicle treated) apoptosis in HCC78 cells was 11% of total cell population. *-p<0.05, **-p<0.01 and ***-p<0.0001 by t-test.

FIG. 6A is a set of six images on the left and a bar graph on the right. The images at left are of a gap at 10 minutes and at 18 hours after ‘scratch’ of cells on a surface. Cells were treated with vehicle, 50 nM crizotinib or 50 nM foretinib as indicated. The right panel is a bar graph depicting the percent of gap closure determined by measuring the length of a straight line drawn across the gap from imaging the same field of view at initiation of treatment and at 18 hours. Scale bar=200 μm

FIG. 6B is a plot depicting the IC₅₀ values for HCC78, PC9 and HCC4011 cells treated with crizotinib, foretinib and erlotinib as indicated. IC₅₀ was determined from non-linear regression curve fit analysis of the dose response curves.

FIG. 6C is a plot depicting the IC₅₀ values for HCC78 cells treated with crizotinib, foretinib, MGCD-265, SGX-523 and JNJ-38877605 as indicated. IC₅₀ was determined from non-linear regression curve fit analysis of the dose response curves.

FIG. 7A are images of immunoblots of lysates from NIH3T3 FIG-ROS and SLC-ROS cells treated with varying concentration of crizotinib and foretinib for 1 hour and probed for phosphorylated and total ROS1.

FIG. 7B top panels are images of plates depicting NIH3T3 FIG-ROS and SLC-ROS colony formation in soft agar after treatment with crizotinib and foretinib as indicated. The bottom panels are bar graphs indicating the number of colonies formed from the plates depicted in the top panels. The values indicate average colony number±SEM from 4 independent wells.

FIG. 8 is a scatter plot of the individual weight distribution of treated tumors from Lines 1 and 2 (shPten tumors) and Lines 3 and 4 (FIG-ROS expressing tumors). Statistical significance was determined by a paired t-test.

FIG. 9A is a bar graph depicting the viability of Ba/F3 FIG-ROS kinase domain mutant cell lines derived from an ENU mutagenesis screen after transfection with siRNA that silences either the ROS1 kinase domain (siROS1-KD) or nontargeting siRNA (siNT). Viability was normalized to that of vehicle (sterile water) transfected cells.

FIG. 9B is a line graph depicting the results of an IL-3 withdrawal assay for Ba/F3 cells transfected with either the unmutated FIG-ROS or the indicated FIG-ROS kinase domain mutant. Total viable cell number was determined by counting cells on the indicated number of days after IL-3 withdrawal.

FIG. 10A is a dose response curve showing the viability of the indicated Ba/F3 FIG-ROS kinase domain mutant cells after 72 hours of exposure to crizotinib. Viability is normalized to vehicle treated cells of the same genotype. Values are the means of three independent experiments with triplicate wells. Error bars indicate the standard error of the mean.

FIG. 10B is a dose response curve showing the viability of the indicated Ba/F3 FIG-ROS kinase domain mutant cells after 72 hours of exposure to foretinib. Viability is normalized to vehicle treated cells of the same genotype. Values are the means of three independent experiments with triplicate wells. Error bars indicate the standard error of the mean.

FIG. 10C is an image of an immunoblot of lysates from wild type and the indicated kinase domain mutants probed for phosphorylated and total ROS1. Tubulin was probed as a loading control.

### SEQUENCE LISTING

SEQ ID NO: 1 is the protein sequence of the kinase domain of Homo sapiens ROS1 (amino acids 1945 to 2222 in the whole molecule).

SEQ ID NO: 2 is a sense primer for SLC34A2.

SEQ ID NO: 3 is an antisense primer for ROS1.

SEQ ID NO: 4 is the FIG-ROS M13Kin3F primer.

SEQ ID NO: 5 is the ROS M13Kin1 Rev primer.

SEQ ID NO: 6 is the M13F primer.

SEQ ID NO: 7 is the M13REV primer.

### DETAILED DESCRIPTION

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

In addition, the materials, methods, and examples are illustrative only and not intended to be limiting. In order to facilitate review of the various embodiments of the disclosure, the following explanations of specific terms are provided:

Aberrant activity of a tyrosine kinase: Inappropriate or uncontrolled activation of a tyrosine kinase, such as ROS1, for example by over-expression, upstream activation (for example, by upstream activation of a protein that affect a tyrosine kinase), and/or mutation (for example a truncation, deletion, insertion and/translocation which increases the activity, such as but not limited to, kinase activity of a tyrosine kinase), which can lead to uncontrolled cell growth, for example in cancer, non-small cell lung cancer and/or cholangiocarcinoma. In some examples, aberrant activity of a tyrosine kinase is a higher rate of kinase activity than the unmutated tyrosine kinase. In some examples, aberrant activity of a tyrosine kinase is a lower rate of kinase activity than the unmutated tyrosine kinase. Other examples of aberrant activity of a tyrosine kinase include, but are not limited to, mislocalization of the tyrosine kinase, for example mislocalization in an organelle of a cell or mislocalization at the cell membrane relative to the unmutated tyrosine kinase.

Administration: To provide or give a subject an agent, such as a composition that targets/inhibits a ROS1 kinase (such as foretinib) by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Amplifying a nucleic acid molecule: To increase the number of copies of a nucleic acid molecule, such as a gene or fragment of a gene, for example a region of a gene that encodes a tumor biomarker, such as a mutant in the ROS1 kinase domain. The resulting products are called amplification products. An example of in vitro amplification is the polymerase chain reaction (PCR). Other examples of in vitro amplification techniques include quantitative real-time PCR, strand displacement amplification (see U.S. Pat. No. 5,744,311); transcription-free isothermal amplification (see U.S. Pat. No. 6,033,881); repair chain reaction amplification (see WO90/01069); ligase chain reaction amplification; gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

A commonly used method for real-time quantitative polymerase chain reaction involves the use of a double stranded DNA dye (such as SYBR Green I dye). For example, as the amount of PCR product increases, more SYBR Green I dye binds to DNA, resulting in a steady increase in fluorescence. SYBR green binds to double stranded DNA, but not to single stranded DNA. In addition, SYBR green fluoresces strongly at a wavelength of 497 nm when it is bound to double stranded DNA, but does not fluoresce when it is not bound to double stranded DNA. As a result, the intensity of fluorescence at 497 nm may be correlated with the amount of amplification product present at any time during the reaction. The rate of amplification may in turn be correlated with the amount of template sequence present in the initial sample. Generally, Ct values are calculated similarly to those calculated using the TaqMan® system. Because the probe is absent, amplification of the proper sequence may be checked by any of a number of techniques. One such technique involves running the amplification products on an agarose or other gel appropriate for resolving nucleic acid fragments and comparing the amplification products from the quantitative real time PCR reaction with control DNA fragments of known size.

Another commonly used method is real-time quantitative TaqMan® PCR (Applied Biosystems). This type of PCR has reduced the variability traditionally associated with quantitative PCR, thus allowing the routine and reliable quantification of PCR products to produce sensitive, accurate, and reproducible measurements of levels of gene expression. The PCR step can use any of a number of thermostable DNA-dependent DNA polymerases, it typically employs a Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used.

Two oligonucleotide primers are used to generate an amplicon typical of a PCR reaction. A third oligonucleotide, or probe, is designed to detect nucleotide sequence located between the two PCR primers. The probe is nonextendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Examples of fluorescent labels that may be used in quantitative PCR include but need not be limited to: HEX, TET, 6-FAM, JOE, Cy3, Cy5, ROX TAMRA, and Texas Red. Examples of quenchers that may be used in quantitative PCR include, but need not be limited to TAMRA (which may be used as a quencher with HEX, TET, or 6-FAM), BHQ1, BHQ2, or DABCYL. TAQMAN® RT-PCR can be performed using commercially available equipment, such as, for example, ABI PRISM 7700® Sequence Detection System (Perkin-Elmer-Applied Biosystems), or Lightcycler (Roche Molecular Biochemicals).

In one embodiment, the 5′ nuclease procedure is run on a real-time quantitative PCR device such as the ABI PRISM 7700® Sequence Detection System. The system includes a thermocycler, laser, charge-coupled device (CCD), camera and computer. The system amplifies samples in a 96-well format on a thermocycler. During amplification, laser-induced fluorescent signal is collected in real time through fiber optic cables for all 96 wells, and detected at the CCD. The system includes software for running the instrument and for analyzing the data.

In some examples, 5′-nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR can be performed using an internal standard. The ideal internal standard is expressed at a constant level among different tissues, and is unaffected by the experimental treatment. RNAs most frequently used to normalize patterns of gene expression are the mRNA products of housekeeping genes.

Amplification of a nucleic acid sequence may be used for any of a number of purposes, including increasing the amount of a rare sequence to be analyzed by other methods. It may also be used to identify a sequence directly (for example, though an amplification refractory mutation system) or as part of a DNA sequencing method.

Antibody: A polypeptide including at least a light chain or heavy chain immunoglobulin variable region which specifically recognizes and binds an epitope of an antigen (such as a mutant ROS1 kinase domain) or a fragment thereof. Antibodies are composed of a heavy and a light chain, each of which has a variable region, termed the variable heavy (VH) region and the variable light (VL) region. Together, the VH region and the VL region are responsible for binding the antigen recognized by the antibody.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, heteroconjugate antibodies (such as, bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

Anti-proliferative activity: An activity of a molecule, for example a small molecule, an inhibitory RNA, and the like, which reduces proliferation of at least one cell type, but which may reduce the proliferation (either in absolute terms or in rate terms) of multiple different cell types (e.g., different cell lines, different species, etc.). In specific embodiments, the anti-proliferative activity of a small molecule, such as an inhibitor of ROS1 kinase will be apparent against cancer cells obtained from a subject that has aberrant ROS1 tyrosine kinase activity, including cells that have aberrant ROS1 activity and one or more mutations that render the cancer susceptible to foretinib and not other tyrosine kinase inhibitors.

Array: An arrangement of molecules, such as biological macromolecules (such as peptides or nucleic acid molecules) or biological samples (such as tissue sections), in addressable locations on or in a substrate. A “microarray” is an array that is miniaturized so as to require or be aided by microscopic examination for evaluation or analysis. In certain example arrays, one or more molecules (such as an antibody or peptide) will occur on the array a plurality of times (such as twice), for instance to provide internal controls. The number of addressable locations on the array can vary, for example from at least one, to at least 2, to at least 3, at least 4, at least 5, at least 6, at least 10, at least 20, at least 30, at least 50, at least 75, at least 100, at least 150, at least 200, at least 300, at least 500, least 550, at least 600, at least 800, at least 1000, at least 10,000, or more. In some examples, arrays include positive and/or negative controls, such as probes that bind housekeeping genes. In particular examples, an array includes nucleic acid molecules, such as oligonucleotide sequences that are at least 15 nucleotides in length, such as about 15-75 or 15-60 nucleotides in length. In particular examples, an array includes oligonucleotide probes or primers which can be used to detect nucleotides that encode tumor biomarker sequences (including RCC biomarkers). In an example, the array is a commercially available array such as Human Genome GeneChip® arrays from Affymetrix (Santa Clara, Calif.).

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays may be computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Binding or stable binding: An association between two substances or molecules, such as the association of an antibody with a peptide, nucleic acid to another nucleic acid, or the association of a protein with another protein or nucleic acid molecule, or the association of a small molecule drug with a protein (such as a tyrosine kinase) or other biological macromolecule. Binding can be detected by any procedure known to one skilled in the art, such as by physical or functional properties. For example, binding can be detected functionally by determining whether binding has an observable effect upon a biosynthetic process such as expression of a gene, DNA replication, transcription, translation, protein activity (including tyrosine kinase activity) and the like.

Biological signaling pathway: A systems of proteins, such as tyrosine kinases, and other molecules that act in an orchestrated fashion to mediate the response of a cell toward internal and external signals. In some examples, biological signaling pathways include tyrosine kinase proteins, such as ROS1, which can propagate signals in the pathway by selectively phosphorylating downstream substrates. In some examples a biological signaling pathway is disregulated and functions improperly, which can lead to aberrant signaling and in some instances hyper-proliferation of the cells with the aberrant signaling. In some examples, disregulation of a biological signaling pathway can result in a malignancy, such as cancer, for example the aberrant activation of a ROS1 kinase such as the formation of a fusion protein comprising ROS1 (including FIG-ROS and SLC-ROS). A ROS1 biological signaling pathway is a signaling pathway, in which ROS1 plays a role, for example by phosphorylation of downstream targets.

Biomarker: Molecular, biological or physical attributes that characterize a physiological or cellular state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination.

A biomarker may be represented by the sequence of a nucleic acid from which it can be derived or any other chemical structure. Examples of such nucleic acids include miRNA, tRNA, siRNA, mRNA, cDNA, or genomic DNA sequences including any complimentary sequences thereof. One example of a biomarker is a DNA coding sequence for a protein comprising one or more mutations that cause amino acid substitutions in the protein sequence. Such a biomarker may be the coding sequence of a particular part of a protein such as the kinase domain of ROS1 comprising nucleic acid mutations that cause one or more of the following amino acid substitutions G2032R, V2098I, G1971E, L1982F, L1982R, C2060G, L1947R or E1935G.

Cancer: A disease or condition in which abnormal cells divide without control and are able to invade other tissues. Cancer cells spread to other body parts through the blood and lymphatic systems. Cancer is a term for many diseases. There are more than 100 different types of cancer in humans. Most cancers are named after the organ in which they originate. For instance, a cancer that begins in the colon may be called a colon cancer. However, the characteristics of a cancer, especially with regard to the sensitivity of the cancer to therapeutic compounds, are not limited to the organ in which the cancer originates. A cancer cell is any cell derived from any cancer, whether in vitro or in vivo. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc.

“Metastatic disease” or “metastasis” refers to cancer cells that have left the original tumor site and migrate to other parts of the body for example via the bloodstream or lymph system. The “pathology” of cancer includes all phenomena that compromise the well-being of the subject. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

Chemotherapeutic agent or Chemotherapy: Any chemical agent with therapeutic usefulness in the treatment of diseases characterized by abnormal cell growth. Such diseases include tumors, neoplasms, and cancer as well as diseases characterized by hyperplastic growth. In one embodiment, a chemotherapeutic agent is an agent of use in treating cancer, such as cancers characterized by aberrant ROS1 activity, including cancers characterized by aberrant ROS1 activity comprising mutations in the kinase domain of ROS1. Such agents include ROS1 inhibitors such as foretinib and crizotinib. Combination chemotherapy is the administration of more than one agent to treat cancer.

Contacting: Placement in direct physical association, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

Diagnostic: Identifying the presence or nature of a pathologic condition, such as, but not limited to cancer, such as cancer caused by aberrant ROS1 activity, including cancer caused by aberrant ROS1 activity that is insensitive to crizotinib and/or sensitive to foretinib. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis.

Domain: any part of polypeptide that can be demonstrated to mediate a particular protein function. For example, the kinase domain of human ROS1 is from amino acid 1945 to amino acid 2222. The kinase domain of mouse ROS1 is from amino acid 1938 to amino acid 2216. In both cases, the numbers are derived from the amino acid in the full-length wild type ROS1 protein.

Effective amount: An amount of agent, such as a tyrosine kinase inhibitor that is sufficient to generate a desired response, such as reduce or eliminate a sign or symptom of a condition or disease, such as cancer, for example cancers expressing an aberrant ROS1 kinase. When administered to a subject, a dosage will generally be used that will achieve target tissue concentrations that has been shown to achieve anti proliferative activity in vitro. In some examples, an “effective amount” is one that treats (including prophylaxis) one or more symptoms and/or underlying causes of any of a disorder or disease, for example cancer, such as a cancer characterized by an aberrant ROS1 kinase. An effective amount can be a therapeutically effective amount, including an amount that prevents one or more signs or symptoms of a particular disease or condition from developing, such as one or more signs or symptoms associated with cancer.

Inhibitor: Any chemical compound, specific for a protein or other gene product that can directly interfere with the activity of a protein, such as a kinase, particularly a ROS1 kinase and more particularly a ROS1 kinase with aberrant activity. An inhibitor can inhibit the activity of a protein either directly or indirectly. Direct inhibition can be accomplished, for example, by binding to a protein and thereby preventing the protein from binding an intended target, such as a receptor. Indirect inhibition can be accomplished, for example, by binding to a protein's intended target, such as a receptor or binding partner, thereby blocking or reducing activity of the protein. Examples of inhibitors of aberrant ROS1 kinase domains include crizotinib and foretinib.

Inhibit: To reduce to a measurable extent, for example, to reduce activity (including aberrant activity) of a protein such as a kinase. In some examples, the kinase activity of a protein is inhibited, for example using a small molecule inhibitor of ROS1 such as crizotinib or foretinib.

Inhibiting or treating a disease: Inhibiting the full development of a disease or condition, for example, in a subject who has or who is at risk for a disease such cancer, for example, a cancer characterized by a ROS1 kinase with aberrant activity. “Treatment” refers to any therapeutic intervention that ameliorates a sign or symptom of a disease or pathological condition. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of metastases, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology. A “therapeutic” treatment is a treatment administered after the development of significant signs or symptoms of the disease and.

[symbol]

Kinase: An enzyme that catalyzes the transfer of a phosphate group from one molecule to another. Kinases play a role in the regulation of cell proliferation, differentiation, metabolism, migration, and survival. A “tyrosine kinase” transfers phosphate groups to a hydroxyl group of a tyrosine in a polypeptide. In some examples, a kinase is a ROS1 tyrosine kinase. Receptor protein tyrosine kinases (RTKs) contain a single polypeptide chain with a transmembrane segment. The extracellular end of this segment contains a high affinity ligand-binding domain, while the cytoplasmic end comprises the catalytic core and the regulatory sequences.

Non-receptor tyrosine kinases, such as ROS1, can be located in the cytoplasm as well as in the nucleus. They exhibit distinct kinase regulation, substrate phosphorylation, and function. A “preferential” inhibition of a kinase refers to an inhibitor that has the characteristic of inhibiting the activity of one kinase, such as ROS1, more it inhibits the activity of a second kinase, such as ALK or another tyrosine kinase.

Mass spectrometry: A method wherein, a sample is analyzed by generating gas phase ions from the sample, which are then separated according to their mass-to-charge ratio (m/z) and detected. Methods of generating gas phase ions from a sample include electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer). Prior to separation, the sample may be subjected to one or more dimensions of chromatographic separation, for example, one or more dimensions of liquid or size exclusion chromatography or gel-electrophoretic separation.

Mutation: A mutation is any difference in a nucleic acid or polypeptide sequence from a normal, consensus or “wild type” sequence. A mutant is any protein or nucleic acid sequence comprising a mutation. In addition a cell or an organism with a mutation may also be referred to as a mutant.

Some types of mutations include point mutations (differences in individual nucleotides or amino acids); silent mutations (differences in nucleotides that do not result in an amino acid changes); deletions (differences in which one or more nucleotides or amino acids are missing); frameshift mutations (differences in which deletion of a number of nucleotides indivisible by 3 results in an alteration of the amino acid sequence. A mutation that results in a difference in an amino acid may also be called an amino acid substitution mutation. Amino acid substitution mutations may be described by the amino acid change relative to wild type at a particular position in the amino acid sequence. For example, mutations in the kinase domain of ROS1 disclosed herein include G2032R, V2098I, G1971E, L1982F, L1982R, C2060G, L1947R and E1935G. G2032R refers to a mutation from a glycine residue at position 2032 of the wild type protein to an arginine residue in the mutant; V2098I refers to a mutation from a valine residue at position 2098 to an isoleucine in the mutant; G1971E refers to a mutation from a glycine at position 1971 to glutamic acid; L1982F refers to a mutation from a leucine at position 1982 to a phenylalanine, etc.

Pharmaceutically acceptable carriers: The pharmaceutically acceptable carriers of use are conventional. Remington's Pharmaceutical Sciences, by E.W. Martin, Mack Publishing Co., Easton, Pa., 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Phospho-peptide or phospho-protein: A protein in which one or more phosphate moieties are covalently linked to one or more of the amino acids making up the peptide or protein. A peptide can be phosphorylated at multiple or single sites. Sometimes it is desirable for the phospho-protein to be phosphorylated at one site regardless of the presence of multiple potential phosphorylation sites. In vivo the transfer of a phosphate to a peptide is accomplished by a kinase. For example a tyrosine kinase such as ROS1 transfers a phosphate to a tyrosine residue of a substrate peptide or protein.

Polypeptide: Any chain of amino acids, regardless of length or posttranslational modification (such as glycosylation, methylation, ubiquitination, phosphorylation, or the like). In one embodiment, a polypeptide is a ROS1 polypeptide. “Polypeptide” is used interchangeably with “protein,” and is used to refer to a polymer of amino acid residues. A “residue” refers to an amino acid or amino acid mimetic incorporated in a polypeptide by an amide bond or amide bond mimetic.

Purified: The term “purified” does not require absolute purity; rather, it is intended as a relative term. Thus, for example, a purified nucleic acid preparation is one in which the protein referred to is more pure than the protein in its natural environment within a cell. For example, a preparation of a protein is purified such that the protein represents at least 50% of the total protein content of the preparation. Similarly, a purified nucleic acid preparation is one in which nucleic acid is more pure than in an environment including a complex mixture compounds, such as a cell or cell extract. The terms “isolated” or “isolating” are interchangeable with the term “purified” or “purifying.”

Sample: A sample, such as a biological sample, is a sample obtained from a plant or animal subject. As used herein, biological samples include all clinical samples useful for detection of mutations in tumor DNA, particularly mutations in the kinase domain of ROS1. Samples include, but not limited to, cells, tissues, and bodily fluids, including tissues that are, for example, unfixed, frozen, fixed in formalin and/or embedded in paraffin. In one example, a sample includes a tissue biopsy obtained from a subject with a tumor.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N₈O₅, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino 5 acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Small molecule: A molecule, typically with a molecular weight less than about 1000 Daltons, or in some embodiments, less than about 500 Daltons, wherein the molecule is capable of modulating, to some measurable extent, an activity of a target molecule such as inhibiting the activity of a kinase, such as a ROS1 kinase with aberrant activity.

Subject: A living multicellular vertebrate organism, a category that includes, for example, mammals and birds. A “mammal” includes both human and non-human mammals, such as mice. In some examples, a subject is a patient, such as a patient diagnosed with cancer, particularly cancer characterized by a ROS1 kinase with aberrant activity, more particularly a cancer characterized by a ROS1 kinase with aberrant activity comprising one or more mutations in its kinase domain.

Substrate: A molecule that is acted upon by an enzyme, such as ROS1. A substrate binds with the enzyme's active site, and an enzyme-substrate complex is formed. In some examples, the enzyme catalyses the incorporation of an atom or other molecule into the substrate, for example a kinase can incorporate a phosphate into the substrate, such as a peptide, thus forming a phospho-substrate.

Tissue: A plurality of functionally related cells. A tissue can be a suspension, a semi-solid, or solid. Tissue includes parts of organs collected from a subject such as the lung, the liver or a portion thereof.

### Methods of Diagnosis

Disclosed herein are methods of predicting if a subject with a cancer characterized by aberrant activity of ROS1 would benefit from treatment with foretinib, more particularly if the subject would benefit from treatment with foretinib as opposed to another tyrosine kinase inhibitor such as crizotinib.

In particular examples, the methods include identifying a nucleic acid mutation in the kinase domain of ROS1 (SEQ ID NO: 1) that results in an amino acid substitution selected from G2032R, V2098I, G1971E, L1982F, L1982R, C2060G, L1947R or E1935G, either alone or in combination. Detection of one or more of the above-identified mutations in a tumor sample from a subject indicates that the subject should be treated with foretinib as opposed to another tyrosine kinase inhibitor, particularly crizotinib.

The disclosed mutations may be identified by any suitable method known in the art. For example, they may be detected by any method of nucleic acid sequencing, through any method involving nucleic acid amplification, by any method of detecting a protein with one or more of the disclosed mutations, or any combination thereof. Examples of these methods are discussed in detail below.

In some embodiments, the expression of the disclosed biomarker is detected in a sample of a tumor obtained from a subject. Tumor samples can include cancer cells. Tumor samples can also include normal tissue adjacent to the tumor. This normal tissue may serve as an internal negative control, especially in the case of assays that detect expression of a biomarker in the context of tissue structure, including immunohistochemistry, in situ hybridization, in situ polymerase chain reaction, or microdissection followed by nucleic acid amplification. It will appreciated by those of skill in the art that any method of obtaining tissue from a subject can be utilized, and that the selection of the method used will depend upon various factors such as the type of tissue, age of the subject, or procedures available to the practitioner. For example, the tissue sample can be obtained by a variety of procedures including, but not limited to, surgical excision, aspiration, or biopsy alone or in combination with each other or other methods.

### Detecting Cancer Biomarkers

The disclosed ROS1 kinase domain mutations can be detected in a sample using any one of a number of methods well known in the art. Nucleic acids such as genomic DNA, particularly tumor genomic DNA can be isolated from a tumor sample from a subject. General methods of nucleic acid isolation are well known to those of skill in the art. Such methods are disclosed in standard textbooks and handbooks of molecular biology and embodied in commercially available kits.

The mutations can be detected through nucleic acid sequencing. Sequencing may be performed on genomic DNA from the tumor through any method known in the art including Sanger sequencing, pyrosequencing, SOLiD® sequencing, massively parallel sequencing, barcoded sequencing, or any other sequencing method now known or yet to be disclosed.

In Sanger Sequencing, a single-stranded DNA template, an oligonucleotide primer, a DNA polymerase, and nucleotides are used. A label, such as a radioactive label or a fluorescent label is conjugated to some of the nucleotides. One chain terminator base comprising a dideoxynucleotide (ddATP, ddGTP, ddCTP, or ddTTP, replaces the corresponding deoxynucleotide in each of four reactions. The products of the DNA polymerase reactions are electrophoresed and the sequence determined by comparing a gel with each of the four reactions. In another example of Sanger sequencing, each of the chain termination bases is labeled with a fluorescent label and each fluorescent label is of a different wavelength. This allows the polymerization reaction to be performed as a single reaction and enables greater automation of sequence reading.

In pyrosequencing, the addition of a base to a single stranded template to be sequenced by a polymerase results in the release of a pyrophosphate upon nucleotide incorporation. An ATP sulfyrlase enzyme converts pyrophosphate into ATP which in turn catalyzes the conversion of luciferin to oxyluciferin which results in the generation of visible light that is then detected by a camera.

In SOLiD® sequencing, the molecule to be sequenced is fragmented and used to prepare a population of clonal magnetic beads (in which each bead is conjugated to a plurality of copies of a single fragment) with an adaptor sequence. The beads are bound to a glass surface. Sequencing is then performed through 2-base encoding.

In massively parallel sequencing, randomly fragmented targeted DNA is attached to a surface through the use of an oligonucleotide adaptor. The fragments are extended and bridge amplified to create a flow cell with clusters, each with a plurality of copies of a single fragment sequence. The templates are sequenced by synthesizing the fragments in parallel. Bases are indicated by the release of a fluorescent dye correlating to the addition of the particular base to the fragment.

In pyrosequencing, massively parallel sequencing or SOLiD® sequencing, an artificial sequence called a barcode may be added to primers used to clone fragmented sequences or to adaptor sequences. A barcode is a 4-10 nucleic acid sequence that uniquely identifies a sequence as being derived from a particular sample. Barcoding of samples allows sequencing of multiple samples in a single sequencing run. (See Craig D W et al, Nat Methods 5, 887-893 (2008) for descriptions and examples of barcodes.) DNA sequencing methods can, but need not, rely on nucleic acid amplification of a nucleic acid encoding a protein such as ROS1 genomic DNA, ROS1 cDNA, or the ROS1 kinase domain.

Additional methods of detecting mutations in nucleic acids include detection through selective nucleic acid amplification of mutant sequences. An example of such a method is the amplification refractory mutation system (ARMS) Newton et al, Nucleic Acids Res 17, 2503-2515 (1989.) This method uses a primer that matches the nucleotide sequence immediately 5′ of the mutation to be tested with the 3′ end of the primer specific for the nucleotide sequence of the mutant. Such a primer will specifically amplify the mutant nucleic acid but not the wild type amino acid. Such reactions may be adapted to real-time PCR based systems such as TaqMan®.

The disclosed mutations may also be identified using a microarray technique. Sequences corresponding to one or more of the disclosed mutants may be plated or arrayed on a microchip substrate. The arrayed sequences are then hybridized to isolated tumor genomic DNA. An array may also be a multi well plate.

The disclosed mutations may also be identified in proteins by, for example, mass spectrometry or antibodies designed to detect proteins with the disclosed mutations.

### Methods of Treatment

Methods of treating a subject with a cancer characterized by an aberrant ROS1 kinase are provided herein. The methods include selecting a subject with a tumor or tumor clone that has an aberrant ROS1 kinase, such as a subject with a cancer likely to have a FIG-ROS fusion, such as a non-small cell lung cancer or cholangiocarcinoma. The subject is then treated with a ROS1 kinase inhibitor such as foretinib, particularly when the kinase domain of the aberrant ROS1 kinase comprises one or more of the following G2032R, V2098I, G1971E, L1982F, L1982R, C2060G, L1947R or E1935G, including the homologs thereof for non-human mammals.

The administration of foretinib can be for either a prophylactic or a therapeutic treatment. When provided prophylactically, foretinib is provided in advance of any symptom. The prophylactic administration of the compounds serves to prevent or ameliorate any subsequent disease process. When provided therapeutically, the compounds are provided at (or shortly after) the onset of a symptom of disease. For prophylactic and therapeutic purposes, foretinib can be administered to the subject in a single bolus delivery, via continuous delivery (for example, continuous transdermal, mucosal or intravenous delivery) over an extended time period, or in a repeated administration protocol (for example, by an hourly, daily or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition. One of skill in the art in light of this disclosure will be able to determine an effective dose of foretinib.

### Kits

A kit is an assemblage of components that may be used in the performance of the method. Use of kits provides advantages to the end user of the method in that the components may have been standardized, the components may have been subject to quality assurance, the components may have been subject to sterilization, or the proportions and characteristics of the various components may have been optimized for maximal efficacy. In addition, a kit may provide the advantage that the components of the kit are obtained from a single source. This in turn makes preparations for the performance of the method as well as troubleshooting problems with the method more efficient. Components may be enclosed in one or more containers appropriate for their storage, such as vials, tubes, bottles, or any other appropriate container. The containers may be further packaged into secondary containers such as boxes, bags, or any other enclosure.

A diagnostic kit may contain reagents such as oligonucleotides configured to perform nucleic acid amplification (including TaqMan® amplification) that specifically recognize mutant nucleic acids that cause amino acid changes such as V2098I, G1971E, L1982F, L1982R, C2060G, L1947R or E1935G in the ROS1 kinase domain. A diagnostic kit may also comprise an array that includes oligonucleotides that detect the disclosed mutations. A diagnostic kit may also contain a set of primers that amplify the kinase domain for sequencing or any other nucleic acid analysis. A diagnostic kit may also comprise antibodies specific for mutant forms of the ROS1 kinase domain, including V2098I, G1971E, L1982F, L1982R, C2060G, L1947R or E1935G.

A kit may further comprise instructions describing how to perform the method. The instructions may be any description of the method that is provided with, referred to by, or otherwise indicated by a component of the kit. The instructions may be communicated through any tangible medium of expression. The instructions may be printed on the package material, printed on a separate piece of paper or any other substrate and provided with or separately from the kit. They may be printed in any language and may be provided in picture form. The instructions may be posted on the internet, written into a software package, or provided verbally through a telephone or by an email conversation or provided as a smart phone application. The instructions may be said to describe how to perform the method if the instructions provide a recipe of how to perform the method, if they refer a user to a publication wherein a description of the method may be found, or in any other way inform any end user of how to perform the method.

### EXAMPLES

The following examples are illustrative of disclosed methods. In light of this disclosure, those of skill in the art will recognize that variations of these examples and other examples of the disclosed method would be possible without undue experimentation.

### Example 1

### Methods

The methods described in this Example 1 were used in the generation of the results described in Examples 2-7 below.

Cell Culture:

The parental Ba/F3 cell line was cultured in RPMI 1640 medium with 10% FBS, L-glutamine, penicillin/streptomycin and 15% WEHI-conditioned media. Ba/F3 cell lines were maintained at densities of 0.5×10⁶ to 1.5×10⁶ per ml of media. FIG-ROS and SLC-ROS Ba/F3 lines were created by infecting the parental Ba/F3 with retrovirus expressing either human FIG-ROS-S (indicated FIG-ROS herein) or SLC-ROS-S (indicated SLC-ROS herein). Stable transfectant lines were sorted for GFP expression on a FACS Aria® flow cytometer. Cells were counted daily using Guava ViaCount® reagent and Guava Personal Cell Analysis® flow cytometer. NIH3T3 cells were cultured in DMEM-High glucose medium supplemented with 10% FCS, L-glutamine and penicillin/streptomycin. Early passage NIH3T3 cells were infected with retrovirus expressing either FIG-ROS or SLC-ROS and sorted for GFP expression as described above for Ba/F3 cells. HCC78 cells were cultured in RPMI 1640 medium with 10% FBS, L-glutamine, and penicillin/streptomycin. This media is referred to as RPMI-10% FBS herein.

Isolation of Primary Liver Progenitor Cells and Murine Cholangiocarcinoma Tumor Cell Line Generation:

The isolation of liver progenitor cells was performed as previously described in Zender et al, Cold Spring Harb Symp Quant Biol 70, 251-261 (2005); incorporated by reference herein). Hepatoblasts of the genotype AlbCre^{+/-}; Isl Kras^{G12D+/-}; p53^{R172H/loxp} were isolated from ED14.5 mouse embryos and retrovirally transduced with either the FIG-ROS fusion, turbo RFP or a short hairpin RNA against Pten (shPten.1522). At 48 hours following transduction, 1×10⁶ cells were resuspended in 25 μl Matrigel and injected sub-capsular into the livers of immune-deficient mice. Upon tumor formation, tumors were explanted and subjected to collagenase digestion. Digests were plated onto gelatin-coated plates. Tumor cells were enriched by either differential trypsinization or cell sorting. Two cell lines derived by this method expressing FIG-ROS were designated lines 3, 4 and 6. Two cell lines derived by this method expressing shPten were designated lines 1 and 2. These four cell lines were further cultured and maintained in DMEM-High glucose medium supplemented with 10% FCS, L-glutamine and penicillin/streptomycin.

Cell Viability Assay:

Cell lines were distributed in 96-well plates and incubated with the indicated concentrations of inhibitor for 72 hours. Adherent cell lines (for example, HCC78, and the murine cholangiocarcinoma (mCC) cell lines described above) were plated 12 to 18 hours before adding inhibitor. Ba/F3 cell lines were plated at 4×10³ cells/well. HCC78 and mCC cell lines were plated at 1.5×10³ cells/well. All cell types were seeded in a final volume of 80 microliters. A 20 microliter volume of five-fold concentrated inhibitor media was added. Cell proliferation was measured using a methanethiosulfonate (MTS)-based viability assay (CellTiter96 Aqueous One® Solution; Promega). A BioTek Synergy 2® plate reader was used to read the absorbance at 490 nm at 30 minutes and 1 hour after addition of MTS. To facilitate comparison between experiments, the raw MTS absorbance of vehicle (0.1% DMSO) treated cells was set to 1 and absorbance from inhibitor treated wells was normalized to the vehicle treated value. These normalized values are used to plot the graphs shown in the Figures. Each experiment had a minimum of 3 wells per condition and the average and SEM was plotted for curve fit analysis. Data were normalized using Microsoft Excel® and further non-linear regression curve fit analysis of the normalized data for determination of IC₅₀ was performed using Graphpad Prism® software.

Plasmid Construction:

The FIG-ROS-S fusion gene was synthesized using the GeneArt® service. FIG-ROS-S is denoted as FIG-ROS herein. FIG-ROS was further subcloned into the retroviral vector, pMSCV-IRES-GFP (pMIG) using the Gateway® Cloning system. SLC-ROS-S (SLC-ROS) was cloned from cDNA made from HCC78 cells. PCR amplification using a sense primer that was specific for the N-terminus of SLC34A2 (5′ CAC CAT GGC TCC CTG GCC TGA ATT GG) (SEQ ID NO: 2) and an anti-sense primer specific for the C-terminus of ROS1 (5′ TTA ATC AGA CCC ATC TCC ATA TCC ACT GTG AGT G) (SEQ ID NO: 3) amplified both SLC-ROS-L and SLC-ROS-S from HCC78 cDNA. The PCR products corresponding to SLC-ROS-L and SLC-ROS-S were gel purified and cloned into the Gateway Cloning system compatible entry vector, pENTR-D/TOPO. They were then further subcloned into the pMIG retroviral vector as described above. The SLC-ROS-L fusion did not transform Ba/F3 after IL-3 withdrawal so all further inhibitor testing was performed with the SLC-ROS-S, indicated as SLC-ROS. A pMSCV-turboRFP-IRES-GFP construct was generated by inserting turboRFP into the pMIG backbone using BglII/XhoI restriction sites. A pMSCV-shPten.1522-SV40-GFP construct was also generated. FIG-ROS point mutations were created using the Quikchange® Site Directed mutagenesis kit according to the manufacturer's protocol.

IL-3 Withdrawal/Transformation Assay:

Parental Ba/F3, pMIG-alone, FIG-ROS (wildtype or ROS1 kinase domain mutant variants) or SLC-ROS expressing Ba/F3 cells (3×10⁶ cells total) were washed three times with 50 ml of RPMI-10% FBS media and re-suspended in 6 mls of fresh RPMI-10% FBS media. The total number of viable cells was counted every other day using a Guava ViaCount® reagent and a Guava Personal Cell Analysis® flow cytometer. If cells grew to cell densities >1.5×10⁶/ml in withdrawal media, the cells were centrifuged and resuspended in fresh media to keep final culture density of 0.5×10⁶/ml.

In Vivo Treatment of Tumor Bearing Mice with Inhibitors:

Foretinib was dissolved in DMSO (373 mg/ml), aliquoted and stored at −80° C. Prior to use, aliquots were thawed and further diluted in 1% Hydroxypropylmethylcellulose/0.2% SDS for a final foretinib concentration of 3.25 mg/ml. Crizotinib was reconstituted in 1% Hydroxypropylmethylcellulose and 0.2% SDS to 3.25 mg/ml, aliquoted and stored at 80° C. until use. Mouse chorangiocarcinoma cell lines expressing either FIG-ROS or shPten were subcutaneously injected into 11 week old female immune-deficient mice. Upon reaching a tumor diameter of 4-6 mm, mice were treated with foretinib (25 mg/kg; Molecular Weight=632.65), crizotinib (25 mg/kg; Molecular Weight=450.34) or vehicle control by oral gavage once daily for 9 consecutive days. Caliper measurements were taken at the time of treatment initiation and 24 hrs after administration of the last dose. Data in waterfall plots is calculated as follows: ((tumor volume T_{end}/tumor volume T₀)−1). Caliper measurements of tumors were taken at T₀ and T_{end}. Tumor volume is calculated as: 0.5*L*Ŵ2(L>W).

Immunoblotting:

Ba/F3 FIG-ROS, Ba/F3 SLC-ROS, HCC78 and mCC cell lines were treated with the indicated concentration of inhibitors for 1-2 hours. For Ba/F3 derived cell lines: after the completion of treatment, 5×10⁶ cells were pelleted, washed once in ice-cold PBS and lysed in 200 μl of cell lysis buffer supplemented with 0.25% deoxycholate, 0.05% SDS, and protease and phosphatase inhibitors. The protein content of the cell lysates was determined and lysates were either used for immunoprecipitation where indicated or extracted with SDS Sample buffer for 15 minutes at 80° C. Proteins were transferred to Immobilon-FL membranes and immunoblotted with antibodies specific for phospho-ROS1 (Cell Signaling Technology (CST) #3078), total ROS1 (CST #3266), phopsho-Shp2 (CST #3751), total Shpt (CST #3752), phospho-Stat3 (CST #9145), total Stat3 (CST #4904), phospho-Erk1/2 (CST #9101), total Erk2 (Santa Cruz #SC-1647), phospho-S6 (CST #4858), total S6 (CST #2216), phospho-Src (CST #2105), total Src (CST #2110), α-tubulin (Sigma T6199), or β-actin (Sigma A1978) as indicated. Images of the blots were generated by the LI-COR Odyssey® imaging system, following the manufacturer's protocol for immunoblot detection with use of IR-dye conjugated secondary antibodies.

Accelerated Cell-Based Mutagenesis Screen:

Ba/F3 cells expressing FIG-ROS were treated overnight with N-ethyl-N-nitrosourea (ENU; 50 μg/ml), pelleted, resuspended in fresh media, and distributed into 96-well plates at a density of 1×10⁵ cells/well in 200 μl complete media supplemented with crizotinib ranging from 500 nM to 2000 nM. The wells were observed for cell growth under an inverted microscope and media color change every two days for one month. The contents of wells exhibiting cell outgrowth were transferred to a 24-well plate containing 2 mL complete media supplemented with crizotinib at the same concentration as in the initial 96-well plate. At confluency, cells in 24-well plates were collected by centrifugation. DNA was extracted from the cell pellets using a DNEasy® Tissue kit (QIAGEN). The FIG-ROS kinase domain was amplified using primers FIG-ROS M13-Kin3F (5′ GTAAAACGACGGCCAGTGCGAGACTAGCTGCCAAGTAC 3′) (SEQ ID NO: 4) and ROS M13-Kin1 REV (5′ CAGGAAACAGCTATGACCGCCATCTTCACCTTCAAAGC 3′) (SEQ ID NO: 5) and the PCR products were bi-directionally sequenced using M13F (5′ GTAAAACGACGGCCAGTG 3′) (SEQ ID NO: 6) and M13Rev (5′ CAGGAAACAGCTATGACC 3′) (SEQ ID NO: 7) primers. The chromatographs were analyzed for mutations using Mutation Surveyor software (SoftGenetics). We note here that we chose to expand clones only from 750 to 1500 nM crizotinib-treated plates since we observed no outgrowth of clones at 2000 and 2500 nM and a 40% frequency at 500 nM crizotinib.

Statistical Analysis:

Where indicated the Student's t-test was used to determine statistical significance. Comparisons with a p value less than 0.05 when were deemed statistically significant. An asterisk in the figures and/or figure legends indicates statistical significance.

### Example 2

### Screen for ROS1 Inhibitors

The FIG-ROS-S (FIG-ROS) fusion protein has been reported in non-small cell lung cancers (NSCLC), glioblastomas, and cholangiocarcinomas. The expression of FIG-ROS in Ba/F3 or NIH3T3 cell lines is sufficient to transform those lines (Gu T L et al, PLoS One 6, e15640 (2011); incorporated by reference herein.) Disclosed herein are the results of using a screen to identify kinase inhibitors that are selectively cytotoxic to FIG-ROS transformed Ba/F3 cells. The screen is described in detail in Tyner J W et al, Cancer Res 73, 285-296 (2013); incorporated by reference herein.

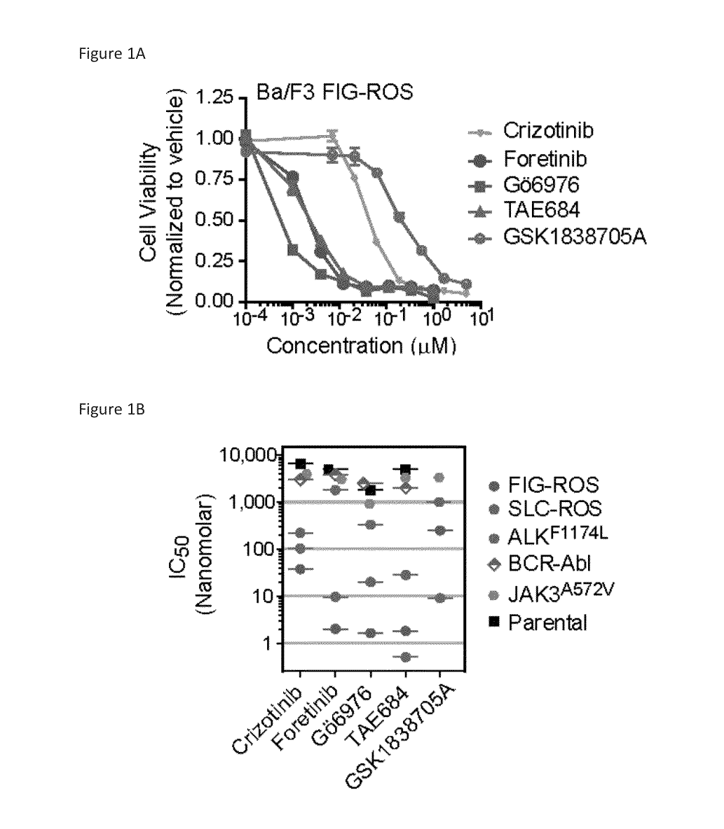

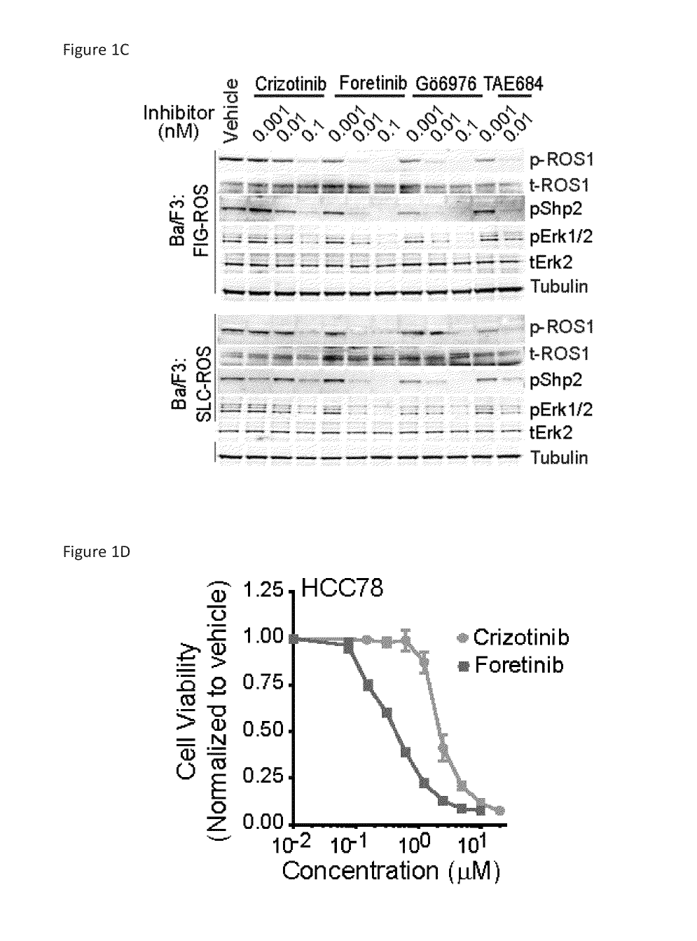

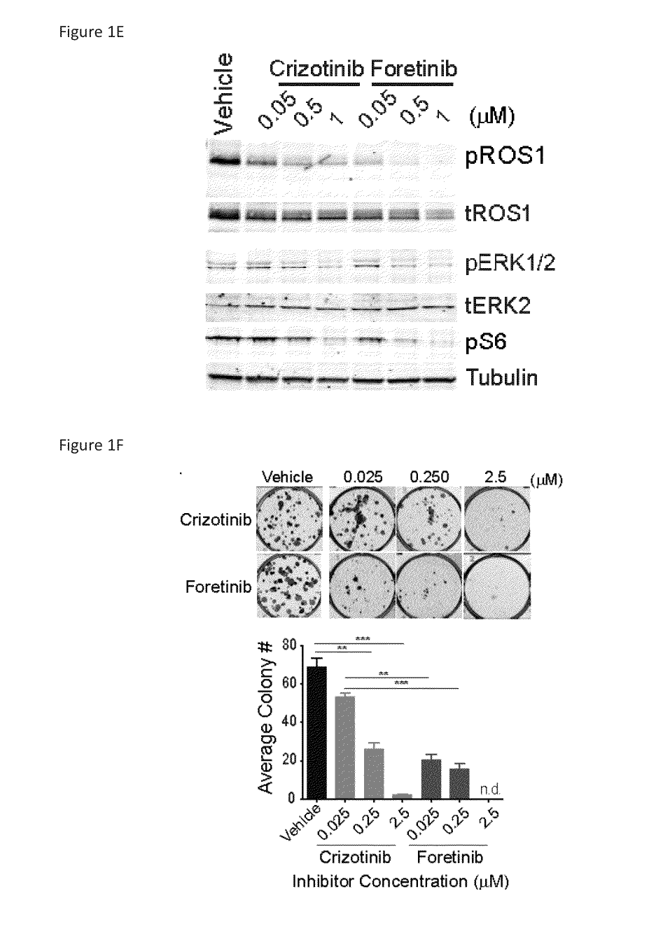

Foretinib (GSK1363089, XL-880) (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide) and Gö6976 (Go6976) (12-(2-cyanoethyl)-6,7,12,13-tetrahydro-13-methyl-5-oxo-5H-indolo(2,3-a)pyrrolo(3,4-c)-carbazole) were shown to be potent ROS1 inhibitors (FIG. 1A) with 50% growth inhibitory concentration (IC₅₀) of 2.2 nM and 1.4 nM respectively. The complete list of inhibitors tested is in Table 1.

TABLE 1
MTS values of compounds tested for ROS1 inhibition-all normalized to an
internal “no drug” control. Foretinib is referred to as XL-880 in this table. Shaded
indicates compounds discussed in the text.

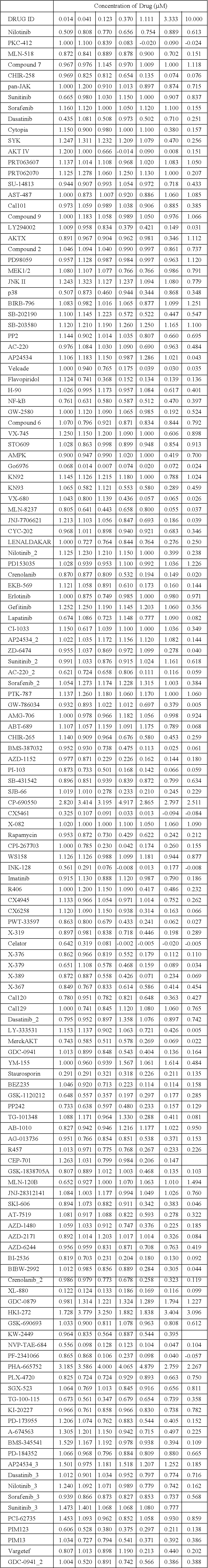

Human ROS1 and anaplastic lymphoma kinase (ALK) share 42-48% amino acid homology in their respective kinase domains, suggesting that ALK tyrosine kinase inhibitors will inhibit ROS1. Recent publications show that the MET/ALK inhibitor, crizotinib (PF-02341066 in Table 1 above), and the ALK inhibitor, TAE684 (NVP-TAE684 in Table 1 above) inhibit the proliferation of Ba/F3 cells transformed by ROS1-fusions as well as the human NSCLC cell line, HCC78, which is characterized by an SLC-ROS fusion (Rikova K et al, Cell 131, 1190-1203 (2007); incorporated by reference herein).

Those results were confirmed by the screen disclosed herein: crizotinib and TAE684 inhibited Ba/F3 FIG-ROS-S cell growth with an IC₅₀ of 38 nM and 2 nM respectively. In addition, Davis et al published the binding affinities (K_{D}) of a total of 72 kinase inhibitors for a total of 442 kinases including ROS1 (Davis M I et al Nature Biotechnology 29, 1046-1051 (2011) and Fabian M A et al, Nature Biotechnol 23, 329-336 (2005); both of which are incorporated by reference herein). In the Davis et al study, the binding affinity of crizotinib (4 nM) for ROS1 is lower than that of foretinib (14 nM). However, in the screen disclosed herein, the IC₅₀ of foretinib (2 nM) for FIG-ROS in a FIG-ROS transformed Ba/F3 cell is 19-fold lower than crizotinib (38 nM). Davis et al (2011) supra reported 19 inhibitors as having disassociation constants of less than 10 μM for ROS1. Of these, crizotinib, TAE684, foretinib and GSK1838705A were reported as having disassociation constants of less than 20 nM.

TABLE 2
Binding affinity of selected inhibitors of the indicated kinases

| | | | | | | | Primary |
|---|---|---|---|---|---|---|---|
| Inhibitor | ROS1 | ALK | MET | PDGFRA | IGF1R | EGFR WT | reported target |
| NVP-TAE684* | 0.49 | 1.1 | 170 | 840 | 2.7 | 180 | ALK |
| Crizotinib* | 4.1 | 3.3 | 2.1 | >10,000 | 780 | >10,000 | MET, ALK |
| Foretinib (XL-880)* | 14 | 69 | 1.4 | 4.5 | 430 | 440 | MET, VEGFR2 |
| GSK1838705A* | 15 | 0.55 | >10,000 | >10,000 | 7 | 4100 | IGF1R, ALK |
| Go6976^{$} | N.A. | N.A. | N.A. | N.A. | N.A. | N.A. | PKC, JAK2, PLK4 |

*Davis et al, 2011 supra
^{$}inhibitory activity for PKC described in Martiny-Baron et al, J Biol Chem 268, 9194-9197 (1993); inhibitory data for JAK2 reported in Grandage et al, Br J Haematol 135, 303-316 (2006); both of which are incorporated by reference herein
N.A.—No data available for binding activity

### Example 3

### Further Selection of Potential ROS-1 Inhibitors

The relative potency of crizotinib, foretinib, Gö6976, TAE684 and GSK1838705A for inhibiting ROS1 and ALK were tested in Ba/F3 expressing FIG-ROS. The results of this experiment are shown in FIG. 1A. In addition, IC₅₀ for the above listed inhibitors were determined in Ba/F3 cells expressing each of the following proteins: cytoplasmic FIG-ROS, membrane associated SLC-ROS, Bcr-Abl, JAK3 comprising an A572V mutation, and ALK comprising a F1174L mutation. The Ba/F3 parental line was also tested. (O'Dwyer M E and Druker B J, Lancet Oncology 1, 207-211 (2000); Walters D K et al, Cancer Cell 10, 65-75 (2006); and Heukamp L C et al, Science Translational Medicine 4, 141ra191 (2012); all of which are incorporated by reference herein.) The results are shown in FIG. 1B. ALK F1174L was relatively insensitive to foretinib (IC₅₀=1800 nM), but foretinib was particularly potent in the cells expressing FIG-ROS (2 nM) and SLC-ROS (10 nM). Both TAE684 and GSK1838705A were effective ALK F1174L inhibitors with IC₅₀ of 0.5 nM and 10 nM respectively. FIG-ROS and SLC-ROS were sensitive to TAE684 with IC₅₀ of 1.8 nM and 28 nM respectively. Both ROS-fusions were relatively insensitive to GSK1838705A with IC₅₀ of 250 nM for FIG-ROS) and 1000 nM for SLC-ROS.

The reported disassociation constants of foretinib and GSK1838705A for ROS1 are 14 nM and 15 nM respectively, yet these compounds have strikingly different inhibitory efficacy for Ba/F3 FIG-ROS cell growth (FIG. 1B). As previously reported in Sasaki T et al, Cancer Res 70, 10038-10043 (2010) ALK F1174L is partially crizotinib-resistant (IC₅₀=103 nM), particularly when compared to TAE684. Ba/F3 Bcr-Abl, JAK3 A572V and parental cells are all insensitive to the crizotinib, foretinib, Gö6976 and TAE684 up to 2000 nM and resistant to GSK1838705A up to 10,000 nM.

Immunoblots in FIG. 1C show a dose dependent decrease in autophosphorylation of FIG-ROS and SLC-ROS as well as ROS1-driven signaling pathways such as phospho-Shp2and phospho-ERK1/2 in Ba/F3 cells when treated with increasing amounts of crizotenib, foretinib, Gö6976, and TAE684. Foretinib and Gö6976 (15 nM) induced apoptosis in Ba/F3 FIG-ROS and SLC-ROS cells but not ALK F1174 cells (FIG. 5A).

### Example 4

### Comparison Between Crizotinib and Foretinib

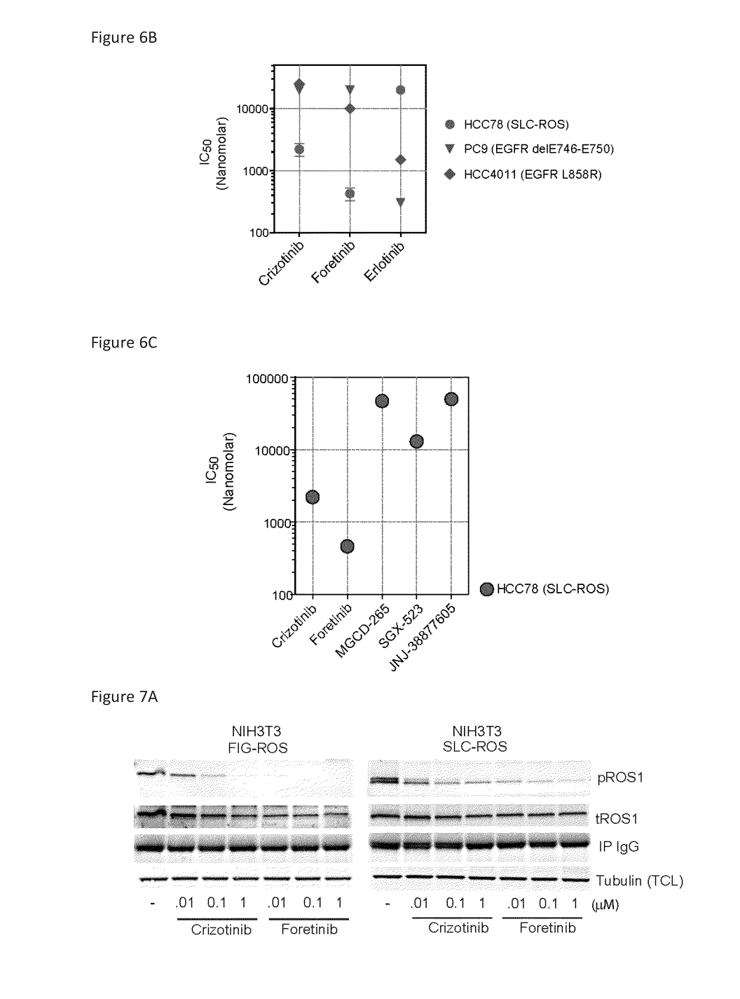

The relative effects of crizotinib and foretinib to suppress the viability of HCC78 cells was compared. The HCC78 line comprises an SLC-ROS mutation. Foretinib inhibited HCC78 cell growth with an IC₅₀ of 425 nM compared to crizotinib (IC₅₀=2200 nM) (FIG. 1D). Furthermore, foretinib inhibited both SLC-ROS and ERK1/2 phosphorylation in HCC78 cells at a lower concentration than did crizotinib (FIG. 1E). In addition, HCC78 colony formation (FIG. 1F) and cell migration (FIG. 6A) were also more potently inhibited by foretinib than crizotinib.

Foretinib and crizotinib activity were also tested using the human non-small cell lung cancer cell lines, PC9 and HCC4011. Cell growth in both lines is driven by activated EGFR and both lines are sensitive to erlotinib. However, both cell lines are relatively insensitive to foretinib and crizotinib (FIG. 6B).

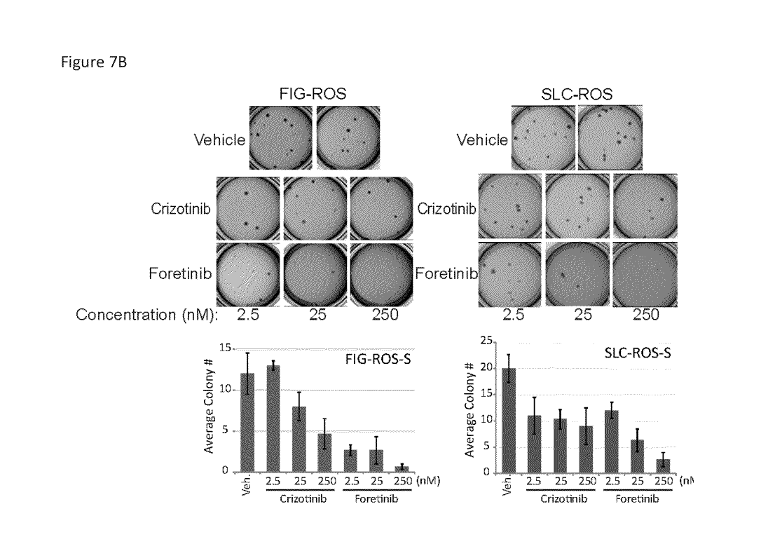

In addition to the SLC-ROS fusion protein described above, the HCC78 cell line also has an activated MET activation. However, the silencing of ROS1 in HCC78 cells using ROS1 specific siRNA results in a loss of cell viability. Therefore, HCC78 cells require SLC-ROS for oncogenic growth. Crizotinib is a dual specificity, MET/ALK inhibitor. As a result, it is possible that the crizotinib sensitivity of HCC78 cells may be due to inhibition of MET and therefore independent of ROS1. (Komiya T et al, Off J Am Soc Clin Oncol 30, 3425-3426 (2012) incorporated by reference herein) Foretinib is also a MET inhibitor. Therefore, it was possible that other MET inhibitors (MGCD-265, SGX-523 and JNJ38877605) could decrease HCC78 cell growth. However, HCC78 cells are resistant to MGCD-265, SGX-523 and JNJ38877605, suggesting that the effect of foretinib and crizotinib in these cells is due to the inhibition of SLC-ROS and not MET (FIG. 6C). Foretinib strongly suppressed FIG-ROS and SLC-ROS phosphorylation and anchorage-independent colony formation at lower doses than crizotinib in NIH3T3 cells transformed with both FIG-ROS and SLC-ROS (FIG. 7A and FIG. 7B).

### Example 5

### Inhibition of FIG-ROS Kinase Fusions in Mouse Cholangiocarcinoma Cell Lines by Foretinib

Cholangiocarcinoma (CC) or cancer of the bile duct is the second most common hepatic malignancy. It is refractory to treatment and has a median survival of less than two years. Recently, FIG-ROS kinase fusions have been reported in 8.7% of cholangiocarcinoma patients, (Gu T L et al, 2011 supra). Murine cholangiocarcinoma tumor cell lines were therefore generated as described in Example 1 above. Pten null murine cholangiocarcinoma tumor cell lines expressing a short hairpin RNA that silences Pten (shPten) were generated as a control.

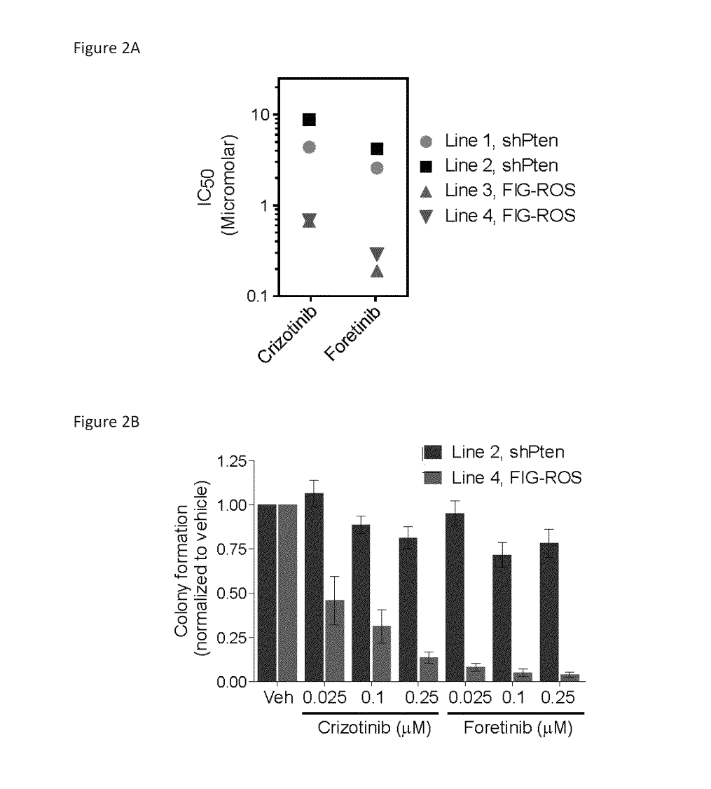

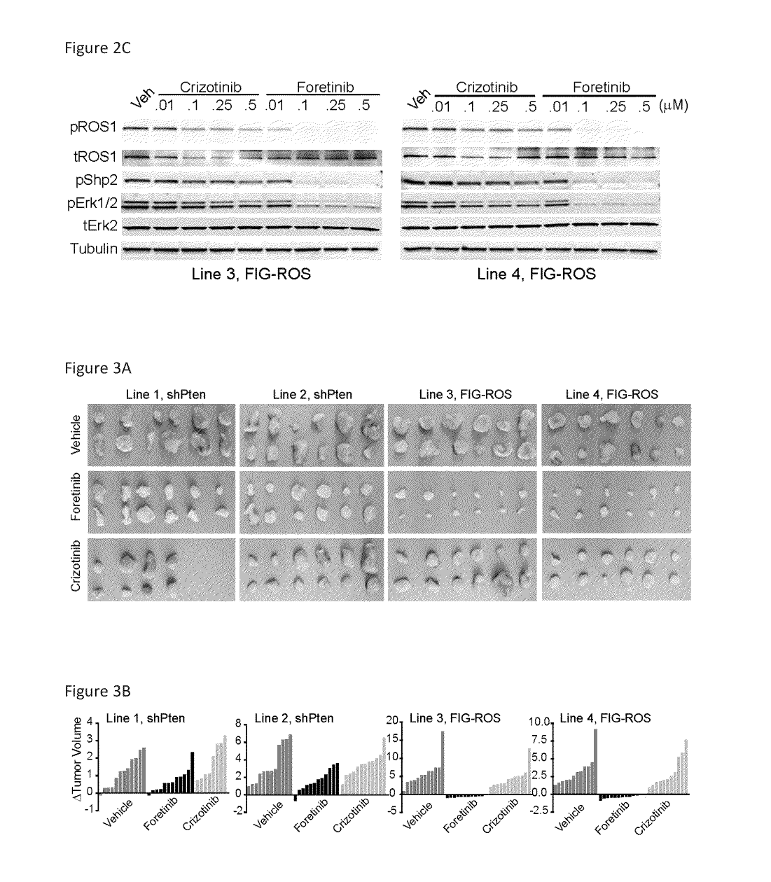

Cell viability assays were used to determine the efficacy and selectivity of crizotinib and foretinib to block cell growth of FIG-ROS (Lines 3, 4, 6) and shPten-expressing (Lines 1, 2) murine cholangiocarcinoma tumor cells. Both foretinib (IC₅₀=300 nM) and crizotinib (IC₅₀=690 nM) inhibited the growth of FIG-ROS but not of shPten cholangiocarcinoma tumor cell lines (IC₅₀>2000 nM) (FIG. 2A). Crizotinib and foretinib also inhibited the ability of FIG-ROS cholangiocarcinoma tumor cells but not shPten cholangiocarcinoma tumor cells to form anchorage-independent colonies in soft agar. The inhibition was dose dependent (FIG. 2B). To directly assess inhibition of FIG-ROS phosphorylation, immunoblot analyses of lysates from vehicle or inhibitor-treated cells were performed. At 100 nM, foretinib almost completely inhibited auto-phosphorylation of FIG-ROS and of phosphorylation of Shpt and ERK1/2. Crizotinib at the same concentration had a more moderate effect on the phosphorylation (FIG. 2C).

### Example 6

### Foretinib Treatment of FIG-ROS Kinase Fusions in Mouse Cholangiocarcinoma Cell Lines In Vivo

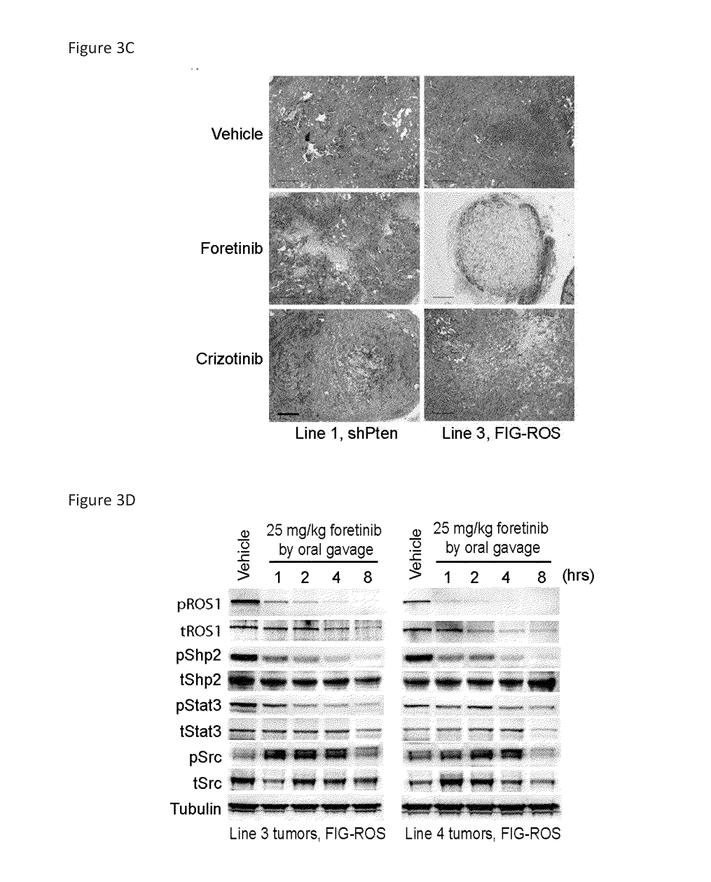

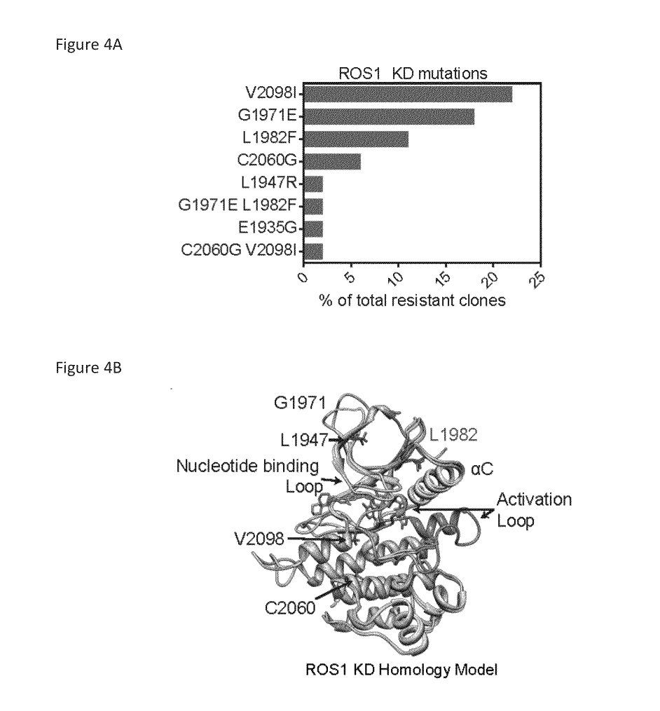

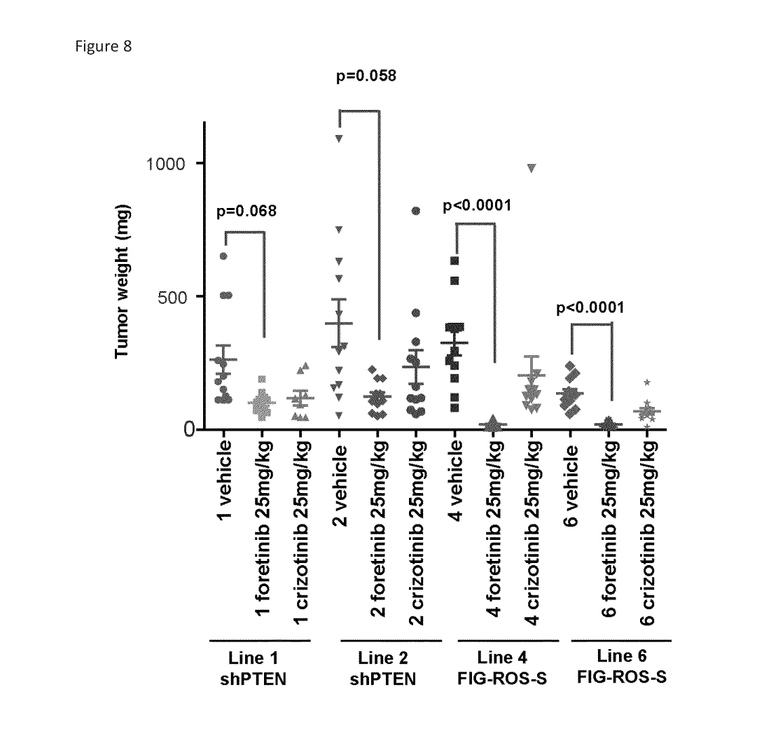

Tumors were formed by subcutaneous injection of the murine cholangiocarcinoma cell lines described above in the flanks of immunodeficient mice. Upon reaching a measurable tumor diameter, the mice were treated with either vehicle, foretinib (25 mg/kg), crizotinib (25 mg/kg) once daily by oral gavage. Caliper measurements were taken at the time of treatment initiation (Tᵢ) and 24 h after administration of the last dose (T_{end}). After 9 days of treatment, the tumors were explanted and measured (FIG. 3A). The waterfall plots down in FIG. 3B, show the effect of inhibitor treatment on relative tumor growth suppression and/or regression as calculated from Tᵢ and T_{end} caliper measurements taken. Foretinib dramatically and significantly reduced FIG-ROS tumor volume (p<0.0001 for Lines 4 and 6) compared to shPTEN tumors (P=0.068 and 0.058 for Lines 1 and 2) (FIG. 3A, FIG. 3B, and FIG. 8). Equal dosing with crizotinib had a moderate but statistically insignificant effect on FIG-ROS tumor volume.

To biochemically assess the efficacy of oral foretinib treatment on intra-tumoral suppression of FIG-ROS and ROS-1 effector pathway phosphorylation, FIG-ROS tumors were harvested from mice treated with vehicle or 25 mg/kg foretinib at 1, 2, 4 and 8 hours after oral gavage and processed for immunoblotting. Rapid and robust inhibition of FIG-ROS phosphorylation was observed in foretinib-treated animals compared to vehicle-treated animals (FIG. 3D). Concomitant with diminished FIG-ROS activation, phosphorylation of Shpt and Stat3, but not Src is inhibited in the treated tumors.

### Example 7

### ROS1 Kinase Domain Mutations that Confer Resistance to Crizotinib

A subset of patients treated with tyrosine kinase inhibitors develop resistance to therapy due to de novo acquisition of kinase domain mutations or other compensatory cellular mechanisms that promote cancer cell growth in presence of inhibitor (Garraway L A and Janne P A Cancer Discovery 2, 214-226 (2012) and Zhang J et al, Nature Rev Cancer 9, 28-39 (2009); both of which are incorporated by reference herein.) Patients with resistant tumors no longer respond to therapy and face poor prognosis unless they can be treated with a secondary agent that suppresses cell growth. For example, second generation Abl kinase inhibitors like dasatinib are successfully used to treat imatinib-resistant CML patients harboring Bcr-Abl KD mutations.

Crizotinib is currently being evaluated in clinical trials as a therapeutic option of ROS1-rerrangments in lung cancer. Partial response of ROS1-fusion bearing patients to crizotinib therapy was recently reported (Shaw A T et al, J Clin Oncol 30, 7508 (2012), incorporated by reference herein) Due to its FDA-approved status, partial therapeutic response and clinical momentum, it is likely that crizotinib will be routinely used to treat lung cancer patients harboring ROS1-fusions in near future. A subset of these ROS1-fusion patients may become resistant to crizotinib therapy, as observed for ALK-fusion lung cancer patients treated with crizotinib (Katayama R et al, Science Translational Med 4, 120ra117 (2012) and Doebele R E et al Clin Cancer Res 18, 1472-1482 (2012); both of which are incorporated by reference herein.)

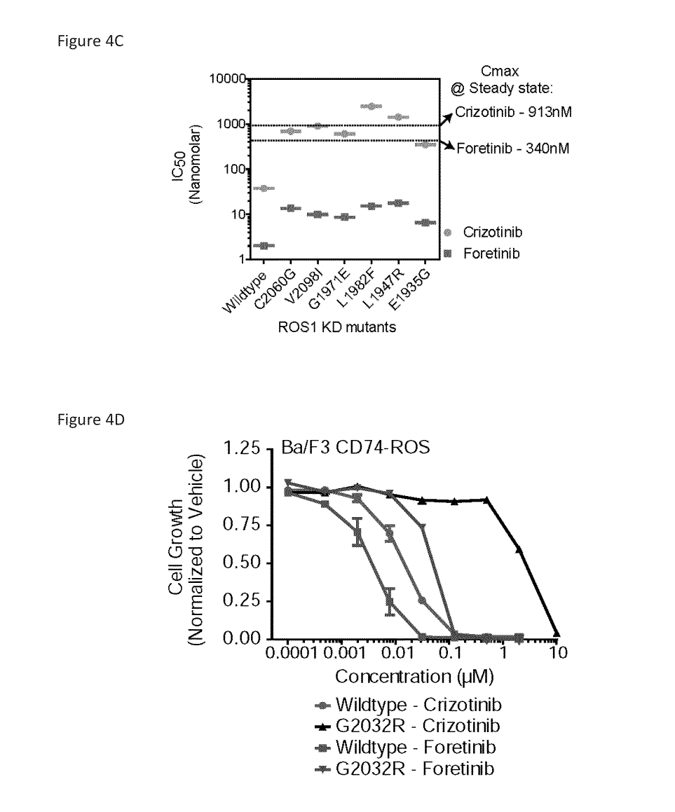

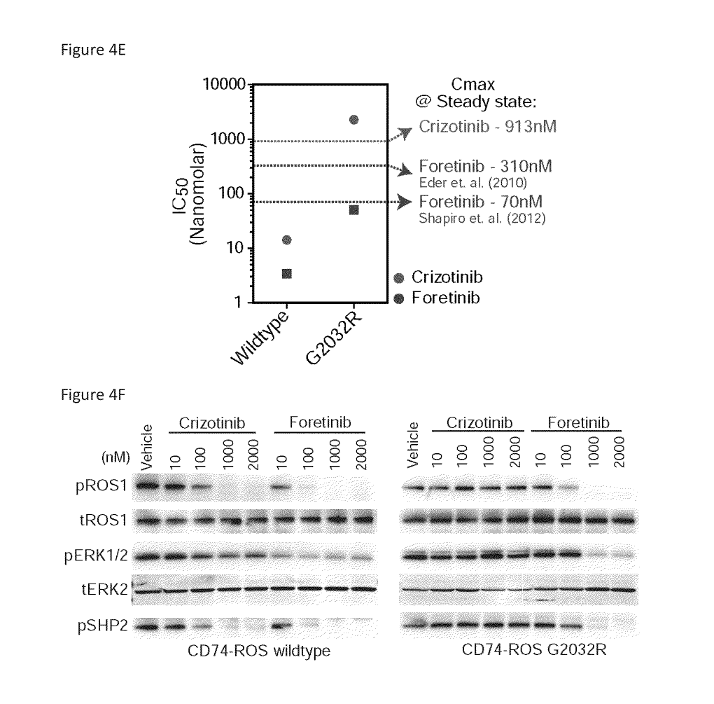

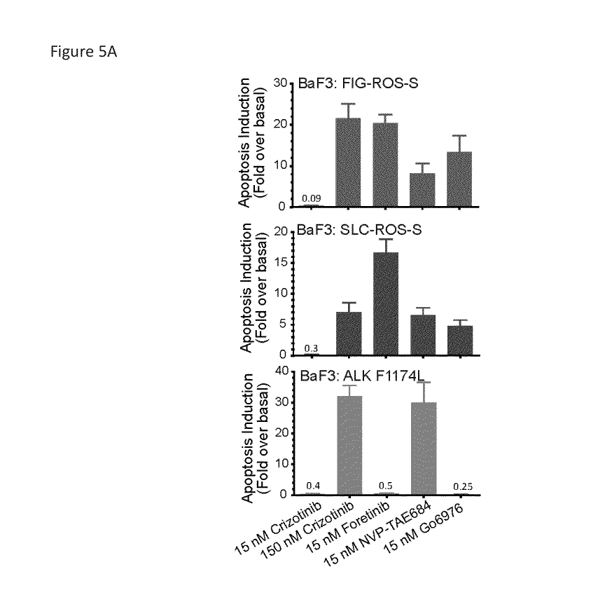

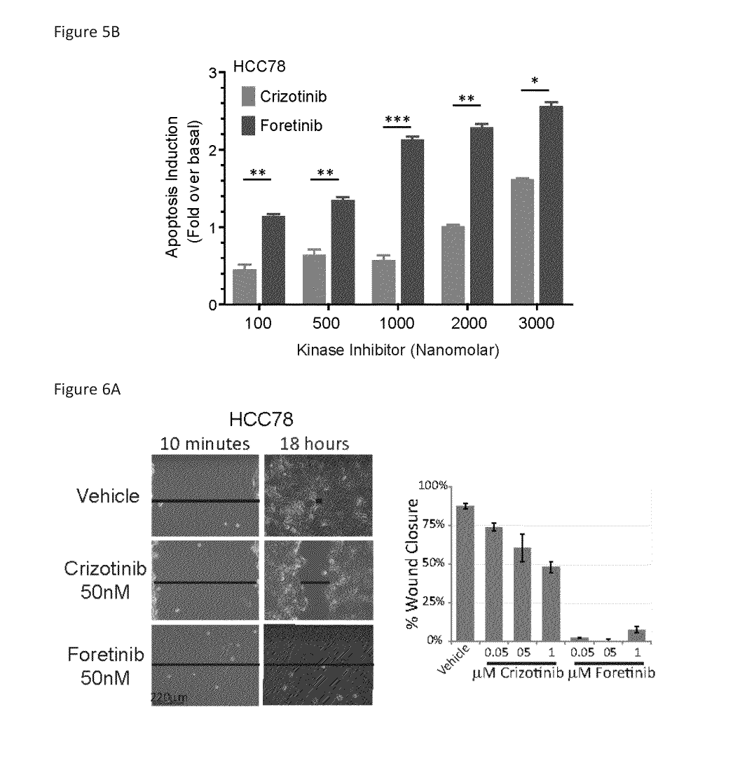

ROS1 kinase domain mutations that confer resistance to crizotinib may be as a predictive strategy for future patient diagnostic and therapy. An N-ethyl-N-nitrosourea (ENU)-assisted accelerated mutagenesis screen was performed using Ba/F3 FIG-ROS cells in the presence of graded concentrations of crizotinib. Multiple ROS1 single and compound kinase domain mutations were found (C2060G, V2098I, G1971E, L1982F, L1974R, E1935G, G1971E/L1982F, C2060G/V2098I) that substantially reduce the efficacy of crizotinib. The frequency of occurrence of these mutations in crizotinib resistant clones is shown in FIG. 4A.

In order to understand how the mutations might confer resistance to inhibition homology models of the kinase domain of ROS1 bound to crizotinib and foretinib were constructed based on crizotinib-bound structure of ALK and foretinib-bound structure of MET. The L1947R, L1982F, and V2098I mutations map close to structural features that are implicated in inhibitor binding (nucleotide binding loop, helix αC, and the activation loop respectively.) Mutations of L1152 in ALK, a residue homologous to L1982 in ROS1, also conferred crizotinib-resistance (Zhang S et al, Chem Biol Drug Design 78, 999-1005 (2011); incorporated by reference herein) and an ALK L1952R mutation was recently identified in a NSCLC patient that had developed clinical resistance to crizotinib Sasaki T et al, Cancer Res 71, 6051-6060 (2011).

### Example 8

### ROS1 Kinase Domain Mutations in FIG-ROS Confer Resistance to Crizotinib

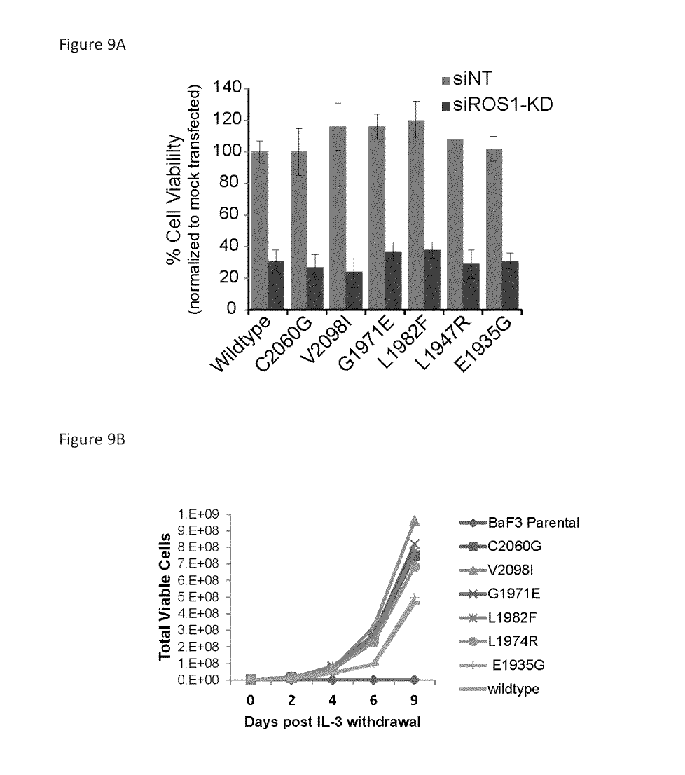

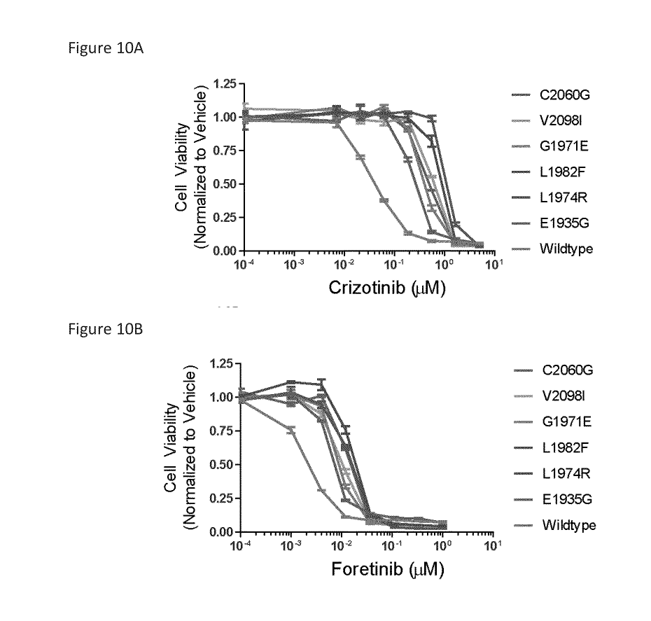

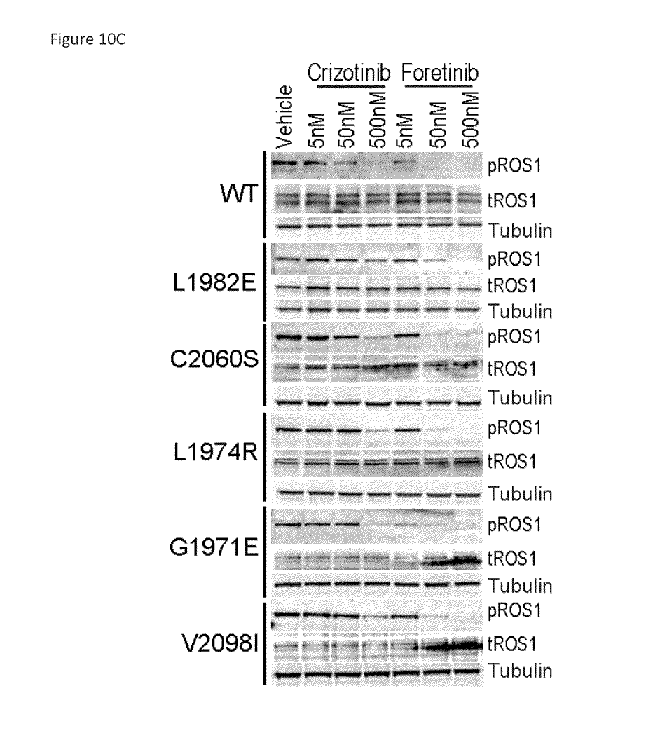

FIG-ROS constructs comprising six of the most frequently recovered crizotinib-resistant mutants (V2098I, G1971E, L1982F, C2060G, L1947R, and E1935G) were generated. All transformed Ba/F3 cells (FIG. 9B). All FIG-ROS mutants exhibited increased resistance to crizotinib (IC₅₀ range 350 to 2450 nM, FIG. 4C and FIGS. 10A, 10B, and Table 3). This is close to the established steady state maximum plasma concentration Cmaxfor crizotinib (411 ng/ml; 913 nM), suggesting that some of the mutations may be inefficiently inhibited at physiologically relevant concentrations.

Notably, while all the tested FIG-ROS mutants also demonstrated decreased sensitivity to foretinib relative to FIG-ROS without a kinase domain mutation, the foretinib IC₅₀ of each mutant was less than 20 nM (FIG. 4C, FIG. 10B, Table 3), which is well below the steady state Cmaxfor foretinib of 340 nM. The differential resistance to crizotinib and foretinib was further evidenced by the reduced phosphorylation of FIG-ROS mutants relative to FIG-ROS without a kinase domain mutation (FIG. 10C)

TABLE 3
IC₅₀ of crizotinib and foretinib for Ba/F3 FIG-ROS kinase domain mutants

| Mutant | Crizotinib IC₅₀ | Foretinib IC₅₀ |
|---|---|---|
| None | 37.5 | 2.03 |
| C2060G | 690 | 13.6 |
| V2098I | 901 | 9.9 |
| G1971E | 605 | 8.69 |
| L1982F | 2450 | 15.27 |
| L1947R | 1420 | 17.87 |
| E1935G | 350 | 6.6 |

### Example 9

### A G2032R Mutation in the ROS1 Kinase Domain Confers Resistance to Crizotinib, but Sensitivity to Foretinib

Therapeutic resistance to crizotinib in a CD74-ROS-rearranged lung adenocarcinoma patient resulting from the acquisition of a G2032R kinase domain point mutation which precludes crizotinib binding due to steric hindrance was reported in Awad M M et al, New Engl J Med 368, 2395-2401 (2013); which is incorporated by reference herein.

To assess whether the G2032R mutation retained sensitivity to foretinib similarly to the ROS1 mutations recovered in the accelerated mutagenesis screen, the efficiency of foretinib to suppress ROS1 phosphorylation and ROS1-dependent cell growth in stably transformed Ba/F3 CD74-ROS wild-type and G2032R mutant cell lines was evaluated. Ba/F3 CD74-ROS^{G2032R} cells were highly resistant to crizotinib (IC₅₀: ^{˜}2200 nM) as compared to wild-type CD74-ROS (IC₅₀: 14 nM) (FIG. 4D). Despite being less sensitive to foretinib than the wildtype CD74-ROS fusion, the CD74-ROS^{G2032R} mutant kinase remained sensitive to foretinib (IC₅₀: 50 nM) at concentrations below plasma levels that can be safely achieved in patients with oral dosing (FIG. 4E). Thus, foretinib may offer an efficient second-line therapy in patients who are treatment refractory due to the acquisition of ROS1 mutations that confer resistance to crizotinib.

1. A method of treating a subject with a cancer characterized by aberrant ROS-1 activity, the method comprising:
receiving a sample comprising a cancer cell from the subject;
isolating a nucleic acid from the sample, wherein the nucleic acid includes a nucleic acid sequence that encodes SEQ ID NO: 1;
identifying a mutation in the nucleic acid encoding SEQ ID NO: 1 that results in an amino acid substitution in G2032, V2098, G1971, L1982, C2050, L1947, or E1935
wherein the presence of one or more of the mutations identifies the sample as comprising a crizotinib resistant and foretinib sensitive cancer cell; and
treating the subject with foretinib.

2. The method of claim 1 wherein the mutation results in an amino acid substitution selected from G2032R, V2098I, G1971E, L1982F, L1982R, C2060G, L1947R or E1935G;

3. The method of claim 1 wherein the cancer cell is a glioblastoma, non small cell lung cancer cell or a cholangiocarcinoma.

4. The method of claim 1 further comprising amplifying the nucleic acid sequence that encodes SEQ ID NO: 1 using the nucleic acid from the sample as a template;

5. The method of claim 1 wherein the mutation is identified by nucleic acid sequencing or polymerase chain reaction.

6. The method of claim 1 wherein the subject is human.

7. The method of claim 6 wherein the cancer cell comprises a ROS1 fusion protein.

8. The method of claim 7 wherein the ROS1 fusion protein is selected from FIG-ROS and SLC-ROS.

9. A kit used to facilitate the performance of the method of claim 1, the kit comprising:
a first set of oligonucleotides configured to amplify the nucleic acid sequence that encodes SEQ ID NO: 1;
a second set of oligonucleotides configured to identify a mutation in SEQ ID NO: 1 that results in an amino acid substitution selected from G2032R, V2098I, G1971E, L1982F, L1982R, C2060G, L1947R or E1935G.

10. The kit of claim 9 wherein the second set of oligonucleotides is configured to form a microarray.

11. The kit of claim 9 further comprising reagents that facilitate nucleic acid sequencing and/or polymerase chain reaction.

12. The kit of claim 9 wherein the first set of oligonucleotides comprises an oligonucleotide that includes SEQ ID NO: 4 and an oligonucleotide that includes SEQ ID NO: 5.
